(19) United States
(12) Patent Application Publication
(10) Pub. No.: US 2023/0352138 A1
(43) Pub. Date: Nov. 2, 2023

(54) Systems and Methods for Adjusting Randomized Experiment Parameters for Prognostic Models

(71) Applicant: Unlearn.AI, Inc., San Francisco, CA (US)

(72) Inventor: Charles Kenneth Fisher, Truckee (CA)

(73) Assignee: Unlearn.AI, Inc., San Francisco, CA (US)

(21) Appl. No.: 18/330,259

(22) Filed: Jun. 6, 2023

Related U.S. Application Data
(63) Continuation-in-part of application No. 18/308,619, filed on Apr. 27, 2023.
(60) Provisional application No. 63/363,795, filed on Apr. 28, 2022.

(51) Int. Cl. G16H 20/10; G16H 50/20

(52) U.S. Cl. CPC G16H 20/10; G16H 50/20

(57) ABSTRACT

Systems and method for estimating treatment effects for a target trial in accordance with embodiments of the invention are illustrated. One embodiment includes a method. The method defines a skedastic function model, wherein defining the skedastic function model depends, at least in part, on target trial data. The method designs trial parameters for the target trial based in part on the skedastic function model. The method applies the trial parameters to a loss function to derive at least one minimizing coefficient, wherein a minimizing coefficient corresponds to a regression coefficient for an expected outcome to the target trial based on the trial parameters. The method computes standard errors for the at least one minimizing coefficient. The method quantifies, using the standard errors, values for uncertainty associated with the target trial. The method updates the trial parameters according to the uncertainty.

### CROSS-REFERENCE TO RELATED APPLICATIONS

The current application is a continuation-in-part of U.S. patent application Ser. No. 18/308,619 entitled “Systems and Methods for Adjusting Randomized Experiment Parameters for Prognostic Models,” filed Apr. 27, 2023, which claims the benefit of and priority under 35 U.S.C. § 119(e) to U.S. Provisional Patent Application No. 63/363,795 entitled “Systems and Methods for Estimating Treatment Effects from Randomized Experiments by Adjusting for Uncertain Prognostic Scores,” filed Apr. 28, 2022, the disclosures of which are hereby incorporated by reference in their entireties for all purposes.

### FIELD OF THE INVENTION

The present invention generally relates to prognostic models and, more specifically, application of prognostic models to assessment of experiment uncertainty.

### BACKGROUND

Randomized Controlled Trials (RCTs) are commonly used to assess the safety and efficacy of new treatments, including drugs and medical devices. In RCTs, subjects with particular characteristics are randomly assigned to one or more experimental groups receiving new treatments or to a control group receiving a comparative treatment (e.g., a placebo), and the outcomes from these groups are compared in order to assess the safety and efficacy of the new treatments.

Prognostic models are mathematical models that relate a subject's characteristics now to the risk of a particular future outcome, thereby allowing for RCTs to be efficiently represented. For example, Artificial Intelligence (AI) and Machine Learning (ML) algorithms may enable prognostic models to use historical data to create more efficient trials without introducing bias. When modelling RCTs in a medical context, prognostic models are used to compute prognostic scores, which correlate to the expected outcome for participants with specific pre-treatment covariates if they receive specific control treatments.

### SUMMARY OF THE INVENTION

Systems and techniques for applying prognostic models to assessment of experiment uncertainty are illustrated. One embodiment includes a method for estimating treatment effects for a target trial. The method defines a skedastic function model, wherein defining the skedastic function model depends, at least in part, on trial data that was applied in a trial. The method designs trial parameters for a target trial based in part on the skedastic function model. The method applies the trial parameters to a loss function to derive at least one minimizing outcome coefficient, wherein the at least one minimizing outcome coefficient corresponds to a regression coefficient for an expected outcome to the target trial based on the trial parameters. The method computes standard errors for the at least one minimizing outcome coefficient. The method quantifies, using the standard errors, values for uncertainty associated with the target trial. The method updates the trial parameters according to the uncertainty.

In a further embodiment, the standard errors are heteroskedasticity-consistent standard errors.

In another embodiment, the expected outcome is obtained through at least one of the group consisting of a digital twin and a prognostic model.

In another embodiment, defining the skedastic function model includes: calculating one or more predicted outcomes for the trial data; obtaining residuals corresponding to the one or more predicted outcomes for the trial data; and using the residuals to define the skedastic function model.

In further embodiment, predicted outcomes for the trial data are based on digital twin outputs.

In a further embodiment, the predicted outcomes are predictions from a regression model fitted on the trial data; and predictors of the regression model are means of the digital twin outputs.

In another further embodiment, the trial data includes participant data for an RCT.

In still another further embodiment, defining the skedastic function model further includes: applying parameters of the skedastic function model to a loss function for data from the target trial, to derive at least one minimizing model coefficient, wherein the at least one minimizing model coefficient includes a treatment effect coefficient; computing standard errors for the at least one minimizing model coefficient; calculating one or more predicted outcomes for the target trial; and defining the skedastic function model further based on variances corresponding to the one or more predicted outcomes for the target trial.

In a further embodiment, predicted outcomes for the target trial are based on digital twin outputs; and minimizing model coefficients are treatment effect coefficients.

In another embodiment, the loss function is a weighted least squares loss function.

In a further embodiment, at least one weight quantity of the weighted least squares loss function is inversely proportional to a predicted variance of outcomes of a participant in the target trial.

In another further embodiment, each weight quantity of the weighted least squares loss function has a positive value.

In yet another further embodiment, at least one weight quantity of the weighted least squares loss function is defined by: implementing, using trial data, an ordinary least squares fit; obtaining least squares coefficients from the ordinary least squares fit; and deriving, from the least squares coefficients and the trial parameters, the at least one weight quantity.

In another embodiment, updating the trial parameters according to the uncertainty includes determining a set of characteristics for the target trial, wherein the set of characteristics includes a number of subjects to be enrolled in each of a control arm and a treatment arm; and the uncertainty is based on at least one of a desired type-I error rate and a desired type-II error rate.

In yet another embodiment, updating the trial parameters includes at least one of: minimizing a total number of samples for at least one selected from the group consisting of a treatment arm of the target trial, a control arm of the target trial, and the target trial in totality; and performing a regression analysis based on the expected outcome.

In a further embodiment, an estimate for coefficients of the regression analysis is represented as: \hat{β}=(Z^{T}Z)⁻¹Z^{T}Y where Y is a vector corresponding to treatment outputs for each participant; and Z is a matrix for which each row (zᵢ) corresponds to a set of predictor variables for a participant (i).

In a further embodiment, the set of predictor variables for each participant include the expected outcome and a corresponding treatment for the participant.

In another further embodiment, minimizing a total number of samples is performed by deriving an expected variance reduction.

In a still further embodiment, deriving the expected variance reduction includes: obtaining a limit for the skedastic function model; deriving a set of estimated variance reductions for the previous trial, wherein the estimated variance reduction for each participant of the previous trial is derived from a ratio between a diagonal entry of a first matrix and a diagonal entry of a second matrix; and determining the expected variance reduction from the set of estimated variance reductions.

In yet another embodiment, Xᵢ is a vector of predictor variables for a participant i; and sᵢ² is a representation of the unknown outcome variance for the participant i. The first matrix is represented as: [symbol] , where:

$${Ω}_{1 / 𝒢 ( {σ}_{i}^{2} )} = E ( \frac{1}{𝒢 ( {σ}_{i}^{2} )} {X}_{i} {X}_{i}^{T} ) , {Ω}_{{s}^{2} / {𝒢 ( {σ}_{i}^{2} )}^{2}} = E ( \frac{{s}_{i}^{2}}{{𝒢 ( {σ}_{i}^{2} )}^{2}} {X}_{i} {X}_{i}^{T} ) ,$$

and [symbol] (σᵢ²) is the limit of the skedastic function model for the participant i. The second matrix is represented as: Ω⁻¹Ωₛ_{₂}Ω⁻¹, where: Ω=E(XᵢXᵢ^{T}), and Ωₛ_{₂}=E(sᵢ²XᵢXᵢ^{T}).

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates uses for generative models in the analysis of clinical trials in accordance with various embodiments of the invention.

FIG. 2 conceptually illustrates an example of a process for designing a randomized controlled trial with prognostic effect estimation.

FIG. 3 conceptually illustrates a process for accounting for historical information while estimating treatment effects from randomized experiments in accordance with certain embodiments of the invention.

FIG. 4 conceptually illustrates a process for Fixed Function Weighted Prognostic Covariate Adjustment in accordance with a number of embodiments of the invention.

FIG. 5 conceptually illustrates a process for Fitted Function Weighted Prognostic Covariate Adjustment in accordance with numerous embodiments of the invention.

FIG. 6 discloses a process for defining skedastic function models in accordance with multiple embodiments of the invention.

FIG. 7 discloses a process for obtaining expected variance reductions in accordance with certain embodiments of the invention.

FIG. 8A illustrates a system for using generative models to estimate treatment effects in accordance with some embodiments of the invention.

FIG. 8B illustrates borrowing information from digital twins to estimate treatment effects in accordance with many embodiments of the invention.

FIG. 9 illustrates using linear models and digital twins to estimate treatment effects in accordance with several embodiments of the invention.

FIG. 10 illustrates a treatment analysis system that determines treatment effects in accordance with some embodiments of the invention.

FIG. 11 illustrates a treatment analysis element that executes instructions to perform processes that determine treatment effects in accordance with various embodiments of the invention.

FIG. 12 illustrates a treatment analysis application for determining treatment effects in accordance with numerous embodiments of the invention.

### DETAILED DESCRIPTION

Turning now to the drawings, systems and methods in accordance with several embodiments of the invention may enable adjustments in various statistical terms and entities including but not limited to treatment effect estimators and/or standard errors. In accordance with some embodiments adjustments may be based on the incorporation of predictor variables including but not limited to prognostic scores determined by models of expected outcomes, and variances of the outcomes. In accordance with many embodiments of the invention, prognostic scores may refer to mean/average values of prognostic models including but not limited to digital twins. Additionally or alternatively, variances of outcomes may refer to variances of prognostic models including but not limited to digital twins. In accordance with many embodiments of the invention, adjustments (e.g., in the form of determined coefficients to the known and/or derived values) may be facilitated through statistical methodology including but not limited to Prognostic Covariate Adjustment (PROCOVA) methods. PROCOVA methodology is disclosed in Unlearn.AI, Inc., “PROCOVA™ Handbook for the Target Trial Statistician.” Ver. 1.0, European Medicines Agency, incorporated herein by reference. In accordance with a number of embodiments of the invention PROCOVA methodology may be implemented using, but is not limited to weighted least squares estimators. Systems implementing such estimators may be referred to as “Fixed Function Weighted PROCOVA” and/or “Fitted Function Weighted PROCOVA” in this disclosure.

Systems and methods in accordance with some embodiments of the invention can determine treatment effects for a randomized controlled trial (RCT) using data sampled from a generative model, design RCTs, and/or determine decision rules for treatments. In this disclosure, randomized controlled trials may also be referred to as experiments and randomized treatments. In developing RCTs, generative Artificial Intelligence (AI) algorithms can be used to generate digital twins, a specific classification of AI-based prognostic models. In this disclosure, digital twins may refer to digital representations of physical objects, processes, services, and/or environments with the capacity to behave like their counterparts in the real world. In the context of drug and medical studies, digital twins can take the form of representations of the range of potential control (placebo) outcomes of particular clinical trial participants given their baseline characteristics.

Additionally or alternatively, data sampled from generative models in accordance with some embodiments of the invention may be referred to as ‘digital subjects’ throughout this description. In many embodiments, digital subjects can be generated to match given statistics of the treatment groups at the beginning of the study. Digital subjects in accordance with numerous embodiments of the invention can be generated for each subject in a study and the generated digital subjects can be used as digital twins for a counterfactual analysis. In various embodiments, generative models can be used to compute a measure of response that is individual to each patient and this response can be used to assess the effect of the treatment. Systems and methods in accordance with several embodiments of the invention can correct for bias that may be introduced by incorporating generated digital subject data.

In certain embodiments, processes in accordance with a number of embodiments of the invention can improve RCT design by reducing the number of subjects required for different arms of the RCT. Processes in accordance with some embodiments of the invention can improve the ability of a system to accurately determine treatment effects from a RCT by increasing the statistical power of the trial. In many embodiments, the process of conducting a RCT can be improved from the design through the analysis and treatment decisions.

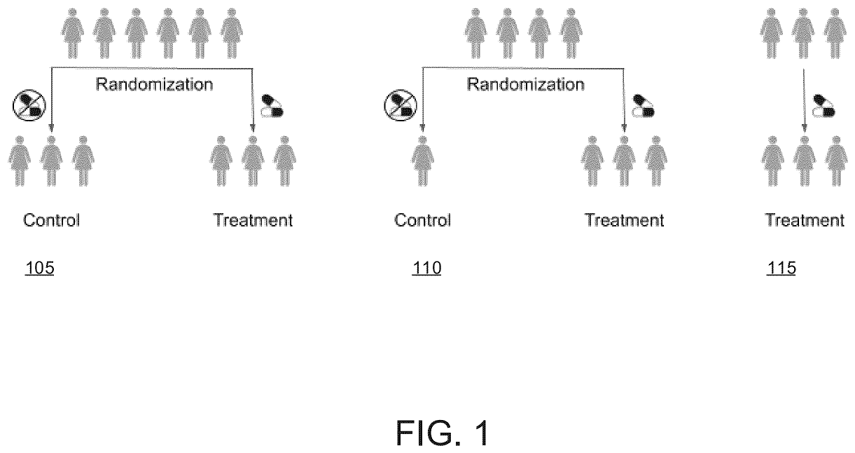

Examples of uses for generative models in the analysis of clinical trials in accordance with various embodiments of the invention are illustrated in FIG. 1. The first example 105 illustrates that generative models, digital subjects, and/or digital twins can be used to increase the statistical power of traditional randomized controlled trials. In the second example 110, generated data is used to decrease the number of subjects required to be enrolled in the control group of a randomized controlled trial. The third example 115 shows that generated can be used as the external comparator arm of a single-arm trial.

In an RCT, a group of subjects with particular characteristics are randomly assigned to one or more experimental groups receiving new treatments and/or to a control group receiving a comparative treatment (e.g., a placebo), and the outcomes from these groups can be compared in order to assess the safety and efficacy of the new treatments. Without loss of generality, an RCT can be assumed to include i=1, . . . , N human subjects. These subjects are often randomly assigned to a control group or to a treatment group such that the probability of being assigned to the treatment group is the same for each subject regardless of any unobserved characteristics. The assignment of subject i to a group is represented by an indicator variable wᵢ. For example, in a study with two groups wᵢ=0 if subject i is assigned to the control group and wᵢ=1 if subject i is assigned to the treatment group. The number of subjects assigned to the treatment group is N_{T}=Σᵢ wᵢ and the number of subjects assigned to the control group is N_{C}=N−N_{T}.

In various embodiments, each subject i in an RCT can be described by a vector xᵢ(t) of variables xᵢⱼ(t) at time t. In this description, the notation X₁={xᵢ(t)}ₜ₌₁^{T} denotes the panel of data from subject i and x_{0,i} to denote the vector of data taken at time zero. An RCT is often concerned with estimating how a treatment affects an outcome yᵢ=ƒ(Xᵢ). The function ƒ(⋅) describes the combination of variables being used to assess the outcome of the treatment. Variables in accordance with a number of embodiments of the invention can include (but is not limited to) simple endpoints based on the value of a single variable at the end of the study, composite scores constructed from the characteristics of a patient at the end of the study, and/or time-dependent outcomes such as rates of range and/or survival times, among others. Approaches in accordance with various embodiments of the invention as described herein can be applied to analyze the effect of treatments on one or more outcomes (such as (but not limited to) those related to the efficacy and safety of the treatment).

Each subject has two potential outcomes. If the subject were to be assigned to the control group wᵢ=0, then yᵢ⁽⁰⁾ would be the observed potential outcome. By contrast, if the subject were to be assigned to receive treatment wᵢ=1, then yᵢ⁽¹⁾ would be the observed potential outcome. In practice, a subject can only be assigned to one of the treatment arms such that the observed outcome is Yᵢ=yᵢ⁽⁰⁾(1−wᵢ)+wᵢyᵢ⁽¹⁾. Potential outcomes in accordance with many embodiments of the invention can include various measurements, such as, but not limited to conditional average treatment effect:

τ(x₀)=E[yᵢ⁽¹⁾|x₀]−E[yᵢ⁽⁰⁾|x₀] (1)

and/or the average treatment effect

τ=E[τ(x₀)]=E[yᵢ⁽¹⁾]−E[yᵢ⁽⁰⁾] (2)

Processes in accordance with several embodiments of the invention can estimate these quantities with high accuracy and precision and/or can determine decision rules for declaring treatments to be effective that have low error rates.

It can be expensive, time-consuming and, in some cases, unethical to recruit human subjects to participate in RCTs. As a result, a number of methods have been developed for using external control arms to reduce the number of subjects required for an RCT. These methods typically fall into two buckets referred to as ‘historical borrowing’ and ‘external control’.

Historical borrowing refers to incorporating data from the control arms of previously completed trials into the analysis of a new trial. Typically, historical borrowing applies Bayesian methods using prior distributions derived from the historical dataset. Such methods can be used to increase the power of a randomized controlled trial, to decrease the size of the control arm, and/or even to replace the control arm with the historical data itself (i.e., an ‘external control arm’). Some examples of external control arms include control arms from previously completed clinical trials (also called historical control arms), patient registries, and data collected from patients undergoing routine care (called real-world data). Use of these external control arms can have serious drawbacks if the population and/or design of the current RCT differs from the population and/or design of the external data sources.

It has recently become possible to apply machine learning methods to create simulated subject records. In addition to data from the RCT, generative models in accordance with several embodiments of the invention can link the baseline characteristics x₀ and the control potential outcome y⁽⁰⁾ through a joint probability distribution p_{θ}_{_{J}}(y⁽⁰⁾,x₀) and a conditional probability distribution p_{θ}_{_{C}}(x₀), in which θ_{J} and θ_{C} are the parameters of the joint and conditional distributions, respectively. Note that a model of the joint distribution will also provide a model of the conditional distribution, but the converse is not true.

In several embodiments, simulated subject records can be sampled from probabilistic generative models that can be trained on various data, such as (but not limited to) one or more of historical, registry, and/or real-world data. Such models can allow one to extrapolate to new patient populations and study designs.

In some embodiments, generative models may create data in a specialized format—either directly or indirectly—such as the Study Data Tabulation Model (SDTM) to facilitate seamless integration into standard workflows. In a variety of embodiments, generating entire panels of data can be attractive because many of the trial outcomes (such as primary, secondary, and exploratory endpoints as well as safety information) can be analyzed in a parsimonious way using a single generative model. For simplicity, the notation p(y, x₀) will be used instead of p(X) in this description, with the understanding that the former can always be obtained from the latter by generating a panel of data X and then computing a specific outcome y=ƒ(X) from the panel.

Systems and methods in accordance with numerous embodiments of the invention can provide various approaches for incorporating data from a probabilistic generative model into the analysis of an RCT. In numerous embodiments, such methods can be viewed as borrowing from a model, as opposed to directly borrowing from a historical dataset. As generative models, from which data can be borrowed, may be biased (for example, due to incorrect modelling assumptions), systems and methods in accordance with a number of embodiments of the invention can account for these potential biases in the analysis of an RCT. Generative models in accordance with various embodiments of the invention can provide control over the characteristics of each simulated subject at the beginning of the study. For example, processes in accordance with various embodiments of the invention can create one or more digital twins for each human subject in the study. Processes in accordance with certain embodiments of the invention can incorporate digital twins to increase statistical power and can provide more individualized information than traditional study designs, such as study designs that borrow population-level information or that use nearest neighbor matches to patients in historical or real-world databases.

### A. Designing Randomized Trials Using Treatment Effect Estimators

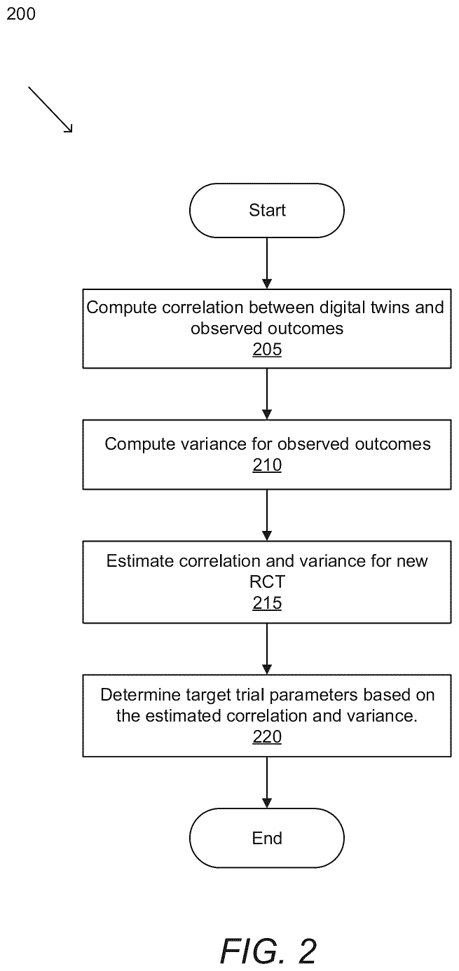

An example of a process for designing a randomized controlled trial with prognostic effect estimation in accordance with an embodiment of the invention is conceptually illustrated in FIG. 2. Process 200 computes (205) a correlation between prognostic scores (or digital twins) and observed outcomes. In various embodiments, prognostic scores can be generated based on subjects from control arms of other trials. In a variety of embodiments, correlations between prognostic scores can include correlations between a vector of outcomes for each sample and prognostic scores generated for the sample. Correlations in accordance with numerous embodiments of the invention can be computed based on an average difference between observed and predicted outcomes. In many embodiments, observed outcomes can come from control arms of other trials. Process 200 computes (210) a variance of the observed outcomes. Variances in accordance with various embodiments of the invention can indicate the unexplained variance between the observed outcomes and the prognostic scores.

Process 200 estimates (215) a correlation and a variance for a new RCT. Estimated correlations and/or variances in accordance with a variety of embodiments of the invention can be based on the correlations and variances for the observed outcomes. In certain embodiments, estimated correlations can be higher than the computed correlations while estimated variances are lower than the computed variances. Processes in accordance with some embodiments of the invention can compute estimated variances based on the computed correlation and/or variance.

Process 200 determines (220) target trial parameters based on the estimated correlation and variance. Target trial parameters in accordance with a number of embodiments of the invention can include (but are not limited to) sample size, control arm size, and/or treatment arm size.

While specific processes for designing random trials are described above, any of a variety of processes can be utilized to design trials as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

Designing randomized trials using treatment effect estimators with frequentist and Bayesian approaches in accordance with some embodiments of the invention are described in greater detail below.

### B. Designing Randomized Trials Using Treatment Effect Estimators

The design of a randomized trial to estimate the effect of a new intervention on a given outcome can depend on various constraints, such as (but not limited to) the effect size one wishes to reliably detect, the power to detect that effect size, and/or the desired control of the type-I error rate. Of course, there may also be other considerations such as time and cost, and one may be interested in more than one particular outcome. Although many of the examples described herein are directed to optimizing for a single outcome, one skilled in the art will recognize that similar systems and methods can be used to optimize across multiple outcomes without departing from this invention.

Treatment effect estimators (or PROCOVA) in accordance with many embodiments of the invention presume a working model Y=β₀+β₁w+β₂μ+∈ where Y, w, and μ are a subject's outcome, treatment status, and prognostic score, respectively and ∈ is a noise term. This model can be fit via ordinary least-squares and the resulting estimate of β₁, represented by \hat{β}₁can be taken as the point estimate of the treatment effect. This estimate is unbiased given treatment randomization without any assumptions about the veracity of the working linear model. Similarly, the estimator of the assumption-free asymptotic sampling variance \hat{ν}² ≡Var[\hat{β}₁] of this estimate is given by:

$${\hat{v}}^{2} = \frac{{\hat{σ}}_{0}^{2}}{{n}_{0}} + \frac{{\hat{σ}}_{1}^{2}}{{n}_{1}} - \frac{{n}_{0} {n}_{1}}{{n}_{0} + {n}_{1}} {( \frac{{\hat{ρ}}_{0} {\hat{σ}}_{0}}{{n}_{1}} + \frac{{\hat{ρ}}_{1} {\hat{σ}}_{1}}{{n}_{0}} )}^{2} \qquad ( 3 )$$

in which \hat{σ}_{W}² is an estimator for the variance term σ_{W}² (representing the variance of the control group when w=0, and the treatment group when w=1); \hat{ρ}_{W} is the estimator of correlation coefficient ρ_{W}; and

$${ρ}_{w} = \frac{Cov [ {Y}_{w} , µ ]}{\sqrt{Var [ µ ] Var [ {Y}_{w} ]}}$$

(where Y_{W} denotes potential outcomes under treatment w=1 and control w=0, σ_{w}²=Var[Y_{w}], while n₀ and n₁ are the number of enrolled control and treated subjects).

An effect estimate can be declared to be “statistically significant” at level α if a p<α where p=2* (min {Φ(\hat{β}₁/\hat{ν}),(1−Φ(\hat{β}₁/\hat{ν}))} is the two-sided p-value, \hat{ν} is the standard error of \hat{β}₁, and Φ denotes the CDF of the standard normal density. The probability that p<α when, in reality, the treatment effect is \hat{β}₁ is given by

$$Power = Φ ( {Φ}^{- 1} ( α / 2 ) + \frac{{β}_{1}}{v} ) + Φ ( {Φ}^{- 1} ( α / 2 ) - \frac{{β}_{1}}{v} ) . \qquad ( 4 )$$

To power a trial to a given level (e.g. 80%) one must first estimate values for σ_{W}² and ρ_{W} using prior data (discussed below) or expert opinion. The power formula can then be composed with the variance formula with σ² and ρ_{W} fixed at their estimates \hat{σ}_{W}² and \hat{ρ}_{W}. The resulting function returns power for any values of n₀ and n₁.

The goal of a sample size calculation in the design of a clinical trial that uses PROCOVA can be to estimate n₀ and n₁ required to achieve the required power. However, one needs an additional constraint such as (but not limited to) a chosen randomization ratio n₀/n₁, or minimizing the total trial size n₀+n₁. In this example, the randomization ratio is pre-specified, but the same principles can be easily applied to other situations.

In numerous embodiments, processes for designing a trial can be based on a generative (or prognostic) model. Prognostic models in accordance with many embodiments of the invention can be trained (e.g., based on a prior trial) or pre-trained. Processes can then estimate the variances, σ_{W}² and correlations, ρ_{W} of the control arm of the trial. One method for obtaining these estimates is to use historical data, such as data from the placebo control arms of previous trials performed on similar populations. In numerous embodiments, estimates can be based on a vector Y′=[Y′₁ . . . Y′_{n′}] of outcomes for these subjects, gathered during the trials, and their corresponding prognostic scores if μ′=[μ′₁ . . . μ′_{n′}], calculated with the prognostic model from each subject's vector of baseline covariates X, i.e. μ′ᵢ=μ(X′ᵢ).

In some embodiments, control-arm marginal outcome variance σ₀² can be estimated with the usual estimator

$${\hat{σ}}_{0}^{2} = \frac{1}{n ′ - 1} \sum {( {Y}_{i}^{′} - {\overline{Y}}^{′} )}^{2}$$

where Y̅′ is the sample average.

The correlation ρ₀ between μ′ and Y′ can be estimated by \hat{ρ}₀=Σ(Y′ᵢ−Y̅′)(μ′ᵢ−μ̅′)/\sqrt{Σ(Y′ᵢ−Y̅′)²Σ(μ′ᵢ−μ̅′)²}, the usual sample correlation coefficient. These values may be inflated (for σ₀²) or deflated (for ρ₀) in order to provide more conservative estimates of power.

In certain embodiments, an inflation parameter λ_{W} for the variance and a deflation parameter γ_{W} for the correlation can be applied to sample size calculation. Inflation and deflation parameters can be used to account for the prognostic model. Define the target effect size β₁^{*}, the significance threshold α, the desired power level ζ, fraction of subjects to be randomized to the active arm π, and dropout rate d. Define γ_{W}≥1 and λ_{W} ∈[0,1] for w=0,1. Define the variance of the potential outcome under active treatment w in the planned trial as γ_{W}²\hat{σ}₀², so that a large γ_{W} inflates the estimated variance. Similarly, define the correlation between the potential outcome and the prognostic model under active treatment w as λ_{W}\hat{ρ}₀, so that a small λ_{W} deflates the estimated correlation. Then n could be minimized using a numerical optimization algorithm (such as a binary search) such that

$$ζ \geq Φ ( {Φ}^{- 1} ( \frac{α}{2} ) + \frac{{β}_{1}^{*}}{v} ) + Φ ( {Φ}^{- 1} ( \frac{α}{2} ) + \frac{{β}_{1}^{*}}{v} ) , with \qquad ( 5 )$$

$${v}^{2} = \frac{1}{n} ( \frac{{γ}_{0}^{2} {\hat{σ}}_{0}^{2}}{1 - π} + \frac{{γ}_{1}^{2} {\hat{σ}}_{0}^{2}}{π} + \frac{{\hat{θ}}^{2} - 2 {\hat{θ}}_{*} \hat{θ}}{π ( 1 - π )} ) ,$$

$$\hat{θ} = {\hat{ρ}}_{0} {\hat{σ}}_{0} ( ( 1 - π ) {λ}_{0} {γ}_{0} + π {λ}_{1} {γ}_{1} ) , and$$

$${\hat{θ}}_{*} = {\hat{ρ}}_{0} {\hat{σ}}_{0} ( {πλ}_{0} {γ}_{0} + ( 1 - π ) {λ}_{1} {γ}_{1} ) .$$

The minimum sample size can be estimated to be

$${n}_{d} = \frac{n}{1 - d} .$$

Unlike the variances and correlations for a control arm, the corresponding values for the treatment arm can rarely be estimated from data because treatment-arm data for the experimental treatment is likely to be scarce or unavailable. In many embodiments, processes can assume σ₀²=σ₁² and ρ₀=ρ₁, the latter of which holds exactly if the effect of treatment is constant across the population. It may also be prudent (and conservative) to assume a slightly higher value for σ₁² and a slightly smaller value for ρ₁ relative to their control-arm counterparts.

With the four parameters σ_{W}² and ρ_{W} specified, the power formula can be computationally optimized over n₀ and n₁ in the desired randomization ratio n₀/n₁ until the minimum values of n₀ and n₁ are found such that the output power meets or exceeds the desired value (e.g., with a numerical optimization scheme).

In many cases, a trial will aim to assess the effect of the intervention on many different outcomes. Processes in accordance with several embodiments of the invention can use multiple prognostic models (e.g., one to predict each outcome of interest) and/or a multivariate prognostic model. Depending on the variances of the outcomes, and the accuracy with which they can be predicted, sample size calculations on the various outcomes of interest may suggest different required sample sizes. In this case, one could simply choose the smallest sample size that meets the minimum required statistical power on each of the outcomes of interest.

### C. Designing Randomized Trials using Bayesian Treatment Effect Estimators

Bayesian PROCOVA is a generalization of PROCOVA that incorporates additional information about the parameters of the (generalized) linear model. Although an example is described with reference to a joint Normal Inverse-Gamma prior distribution, one skilled in the art will recognize that similar processes may utilize various other choices for prior distributions for these parameters without departing from the invention.

In this example, Bayesian PROCOVA specifies a joint Normal Inverse-Gamma prior distribution for the unknown parameters:

$${σ}^{2} ∼ Inverse - Gamma ( ϵ , ϵ ) \qquad ( 6 )$$

$$\frac{1}{σ} ( \begin{matrix}{β}_{0} \\ {β}_{1} \\ {β}_{2}\end{matrix} ) \cdot N ( 0 , ( \begin{matrix}{λ}^{2} & 0 & 0 \\ 0 & 1 / ϵ & 0 \\ 0 & 0 & 1 / ϵ\end{matrix} ) ) . \qquad ( 7 )$$

Here, ∈ is set to an arbitrary small number (e.g., 10⁻⁴) to encode diffuse prior distributions over σ², β₁/σ, and β₂/σ. In certain embodiments, an informative prior can be placed over the ratio β₀/σ to express the belief that β₀/σ∨≤λ_{β}. Values for λ_{β}in accordance with a variety of embodiments of the invention may be elicited by reviewing the predictive performance of the prognostic model on historical trials.

In this formulation, σ₂ is the residual variance that isn't explained by the prognostic model. That is, the notation in this section can be linked to the previous through the relation σ₂=σ_{W}²(1−ρ_{W}²) for w=0,1, which assumes that the unexplained variance is the same in the control and treatment groups.

There are many potential methods to elicit λ_{β}from historical data. In one example, previous clinical trials with similar inclusion criteria to the target trial can be identified. For instance, if the target trial includes only patients with baseline scores on a given diagnostic test that lie within a specified range, past trials where at least P % (e.g., P=90%) of subjects have baseline scores within that range can be assembled. These past trials can be enumerated as j=1, . . . , m. Then for each past trial, j, in the reference set, the pairs (Y_{i,j}, M_{i,j}) can be extracted across all control subjects, i. Define Nⱼ as the sample size for each past trial, and set Eⱼ=\hat{β}_{0,j}/\hat{σ}ⱼ, where:

$${\hat{β}}_{0 , j} = \frac{1}{{N}_{j}} {\sum }_{j} ( {Y}_{i , j} - {M}_{i , j} ) \qquad ( 8 )$$

$${\hat{σ}}_{j}^{2} = \frac{1}{{N}_{j}} {\sum }_{i} {( {Y}_{i , j} - {M}_{i , j} - {\hat{β}}_{0 , j} )}^{2} . \qquad ( 9 )$$

Finally, choose:

$${λ}_{B} = \sqrt{\frac{{\sum }_{j} {E}_{j}^{2}}{{( {F}_{m}^{{χ}^{2}} )}^{- 1} ( γ )}} , \qquad ( 10 )$$

in which Fₘ^{x}^{²} denotes the cumulative distribution function of a [symbol] ₘ² random variable, and γ∈(0,1) is chosen to reflect quantile of the distribution. For example, setting γ=0.025 ensures that it's likely that that |β₀/σ|<λ.

Under the linear model used for PROCOVA, β₁ can be identified as the treatment effect. It has a Student-t posterior distribution, whose parameters depend on the observed trial data. In numerous embodiments, Bayesian PROCOVA can use a posterior probability-based decision rule to conclude that an effect is “statistically significant” at level α if the posterior assigns probability exceeding (1−α/2) to either one of the following events: β₁<0 or β₁>0.

The power of this particular Bayesian decision rule can be given by:

$$Power = Φ ( {Φ}^{- 1} ( α / 2 ) \sqrt{\frac{{V}_{11}}{\hat{V}} ( 1 + \frac{( 1 - p ) {β}_{0}^{2}}{{σ}^{2} ( n {λ}^{2} ( 1 - p ) + 1 )} )} + \frac{τ}{σ \sqrt{\hat{V}}} ) + Φ ( {Φ}^{- 1} ( α / 2 ) \sqrt{\frac{{V}_{11}}{\hat{V}} ( 1 + \frac{( 1 - p ) {β}_{0}^{2}}{{σ}^{2} ( n {λ}^{2} ( 1 - p ) + 1 )} )} + \frac{τ}{σ \sqrt{\hat{V}}} ) \qquad ( 11 )$$

in which n=n₀+n₁ is the total sample size, p=n₁/n is the proportion of subjects assigned to the new intervention, and

$${V}_{11} = \frac{n {λ}^{2} + 1}{n ( n {λ}^{2} p ( 1 - p ) + p )} , \qquad ( 12 )$$

$$τ = {β}_{1} + ( \frac{1}{n {λ}^{2} ( 1 - p ) + 1} ) {β}_{0} , \qquad ( 13 )$$

$$V = \frac{p + ( 1 - p ) {( n {λ}^{2} + 1 )}^{2}}{{np ( n {λ}^{2} ( 1 - p ) + 1 )}^{2}} . \qquad ( 14 )$$

The goal of a sample size calculation in the design of a clinical trial that uses Bayesian PROCOVA is to estimate n₀ and n₁ required to achieve the required power. However, one needs an additional constraint such as a chosen randomization ratio n₀/n₁, or minimizing the total trial size n₁+n₁. For this example, the randomization ratio is pre-specified, but the same principles can be easily applied to other situations.

To perform the sample size calculation, processes in accordance with many embodiments of the invention can estimate the true values of β₀ and σ², as well as a pre-specified power to detect a given effect size β₁. In principle, both β₀ and σ² can be estimated from the performance of the prognostic model on historical data. Specifically, β₀ may be the average difference between the observed and predicted outcomes, and σ²=σ₀²(1−ρ₀²) is the unexplained variance. In many cases, however, processes may set β₀=0 when performing a sample size calculation. Finally, given values for β₀ and σ² and the elicited prior distribution, a numerical optimization process can be used to compute the minimum n₀ and n₁ that offer the desired power, subject to a desired constraint on the randomization ratio.

### D. Determining Treatment Effects

Systems and methods configured in accordance with various embodiments of the invention may be directed to determining treatment effects of RCTs. RCT data can include panel data collected from subjects of a RCT. RCT data in accordance with a variety of embodiments of the invention can be divided into control and treatment arms based on whether subjects received a treatment. In many embodiments, RCT data can be supplemented with generated subject data. Generated subject data in accordance with a number of embodiments of the invention can include (but are not limited to) digital subject data and/or digital twin data.

In several embodiments, processes can receive historical data that can be used to pre-train generative models and/or to determine a prior distribution for Bayesian analyses. Historical data in accordance with numerous embodiments of the invention can include (but are not limited to) control arms from historical control arms, patient registries, electronic health records, and/or real-world data.

Digital subject data may be generated using generative models. Generative models in accordance with certain embodiments of the invention can be trained to generate potential outcome data based on characteristics of an individual and/or a population. Digital subject data in accordance with several embodiments of the invention can include (but are not limited to) panel data, outcome data, etc. In numerous embodiments, generative models can be trained directly on a specific outcome p(y|x₀). For example, if a goal of using the generative model is to increase the statistical power for the primary analysis of a randomized controlled trial then it may be sufficient (but not necessary) to only use a model of p(y|x₀).

Alternatively, or conjunctively, generative models may be trained to generate panel data that can be used in the analysis of a clinical trial. Data for a subject in a clinical trial are typically a panel; that is, it describes the observed values of multiple characteristics at multiple discrete timepoints (e.g. visits to the clinical trial site). For example, if a goal of using the generative model is to reduce the number of subjects in the control group of the trial, or as an external comparator for a single-arm trial, then generated panel data in accordance with many embodiments of the invention can be used to perform many or all of the analyses of the trial.

In several embodiments, generative models can include (but are not limited to) traditional statistical models, generative adversarial networks, recurrent neural networks, Gaussian processes, autoencoders, autoregressive models, variational autoencoders, and/or other types of probabilistic generative models. For example, processes in accordance with several embodiments of the invention can use sequential models such as (but not limited to) a Conditional Restricted Boltzmann Machine for the full joint distribution of the panel data, p(X), from which any outcome can be computed.

Systems and methods in accordance with numerous embodiments of the invention may determine treatment effects for RCTs using generated digital subject data. Generative models in accordance with many embodiments of the invention can be incorporated into the analysis of an RCT in a variety of different ways for various applications. In many embodiments, generative models can be used to estimate treatment effect by training separate generative models based on data from the control and treatment arms. Processes in accordance with many embodiments of the invention can use generative models to generate digital subjects to supplement a control arm in an RCT. In certain embodiments, processes can use generative models to generate digital twins for individuals in the control and/or treatment arms. Generative models in accordance with numerous embodiments of the invention may be used to define individualized responses to treatment. Various methods for determining treatment effects in accordance with various embodiments of the invention are described in greater detail herein.

In several embodiments, treatment effects can be determined by fitting generalized linear models (GLMs) to the generated digital subject data and/or the RCT data. In a number of embodiments, multilevel GLMs can be set up so that the parameters (e.g., the treatment effect) can be estimated through maximum likelihood or Bayesian approaches. In a frequentist approach, one can test the null hypothesis β₀=0, whereas the Bayesian approach may focus on the posterior probability Prob(β₀≥0|data,prior).

While specific processes for determining treatment effects in an RCT are described above, any of a variety of processes can be utilized to determine treatment effects as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### E. Determining Treatment Effects by Adjusting for Unknown Prognostic Data

In accordance with many embodiments of the invention, RCTs may be represented by variables including but not limited to prospective outcomes, indicators of behaviors used in the trial, and pre-behavior covariates (i.e., any variables not affected by the behaviors). As indicated above, behaviors may include but are not limited to medical treatments in RCTs. Systems and methods configured in accordance with numerous embodiments of the invention may, prior to initialing certain RCTs, obtain training datasets (Yⱼ, Xⱼ, wⱼ=0) and/or train probabilistic models p(Yⱼ|Xⱼ, wⱼ=0) to predict the outcome for a participant j when they receive the control treatment. As such RCTs configured in accordance with numerous embodiments of the invention may defined by the tuple (Yᵢ, Xᵢ,wᵢ) in which i represents an individual participant to an RCT, Yᵢ is an outcome of interest, Xᵢ is a vector of pre-treatment covariates, and wᵢ is a treatment indicator. In this disclosure, such modeled distributions may be referred to as “prognostic models,” “treatment effect estimators,” “estimators,” and/or “treatment estimators.”

Systems and methods configured in accordance with various embodiments of the invention may be used to facilitate adjustments in treatment effect estimators and/or the standard errors of the treatment effect estimators. Possible prognostic models may include but are not limited to digital twins and digital subjects. Systems may incorporate predictor variables including but not limited to prognostic scores and variance in outcome derived from prognostic models. Systems may be used to obtain unbiased treatment estimators in which uncertainty is minimized. Additionally or alternatively, systems may be configured to account for varying information and precision in outcomes. In doing so, systems in accordance with a number of embodiments may be used to reduce the variance required by estimators and/or sample size required in RCTs.

### 1. General Implementation

As indicated above, in accordance with numerous embodiments of the invention, prognostic models can be used to compute prognostic scores:

μⱼ:=∫yp (y|Xⱼ, Wⱼ=0)dy (A)

to represent the expected outcome(s) for participant j with pre-treatment covariates Xⱼ when/if they receive the control treatment(s). In accordance with a number of embodiments of the invention, when outcomes are continuous, prognostic models can estimate treatment effects from RCTs using linear models and/or least squares functions. Additionally or alternatively, treatment effects may be derived by minimizing loss functions:

$$L ( α , b , τ ) := {\sum }_{i} {( {Y}_{i} - α - b {μ}_{i} - τ {w}_{i} )}^{2} . \qquad ( B )$$

wherein α, b, and τ0 represents regression coefficients and β=(α*, b*, τ*) may be used to represent (minimized) regression coefficients. Here, τ is identified with the treatment effect. The variance of the treatment effect estimators obtained by minimizing the loss function in Formula (B) may be reduced compared to the treatment effect estimator that does not incorporate the prognostic scores in the analysis (“unadjusted analysis”). The percentage reduction in the variance can be because the loss function in Formula (B) incorporates the prognostic scores, and/or that the prognostic scores can explain the variation in the observed outcomes due to the correlation between the prognostic scores and the outcomes. Specifically, the ratio of the variance of the treatment effect estimator obtained from Formula (B) compared to the variance of the treatment effect estimator obtained from the unadjusted analysis may be roughly proportional to the squared correlation coefficient between the prognostic scores and the outcomes. This impact on the variance of the treatment effect estimators is expounded upon in Schuler, A. et al. (2021) “Increasing the efficiency of randomized trial estimates via Linear Adjustment for a prognostic score,” The International Journal of Biostatistics, 18(2), pp. 329-356. Available at: https://doi.org/10.1515/ijb-2021-0072, incorporated by reference in its entirety.

RCTs configured in accordance with many embodiments of the invention may apply treatments (i.e., wᵢ) randomly. Additionally or alternatively, the treatments may be applied in a manner independent to both pre-treatment covariates (i.e., Xᵢ) and the potential outcomes. In accordance with certain embodiments, potential outcomes may include but are not limited to yᵢ⁽⁰⁾, which can represent the potential outcome(s) when participant i is assigned to a control group, and yᵢ⁽¹⁾, when participant i is assigned to a treatment group. As indicated above, treatment effect estimators (or PROCOVA) in accordance with many embodiments of the invention presume a working model Yᵢ=β₀+β₁wᵢ+β₂μᵢ+∈ᵢ where Yᵢwᵢ, and μᵢ are a subject's outcome, treatment status, and prognostic score, respectively and ∈ᵢ is a noise term. This model can be fit via ordinary least-squares and the value of β₁ can be taken as the point estimate of the treatment effect, \hat{β}₁. This estimate is unbiased given treatment randomization without any assumptions about the veracity of the working linear model. Similarly, the estimator of the assumption-free asymptotic sampling variance \hat{ν}²≡Var[\hat{β}₁] of this estimate can be given by:

$${\hat{v}}^{2} = \frac{{\hat{σ}}_{0}^{2}}{{n}_{0}} + \frac{{\hat{σ}}_{1}^{2}}{{n}_{1}} - \frac{{n}_{0} {n}_{1}}{{n}_{0} + {n}_{1}} {( \frac{{\hat{ρ}}_{0} {\hat{σ}}_{0}}{{n}_{1}} + \frac{{\hat{ρ}}_{1} {\hat{σ}}_{1}}{{n}_{0}} )}^{2} \qquad ( C )$$

in which \hat{σ}_{W}² is an estimator for the variance term σ_{W}² (representing the variance of the control group when w=0, and the treatment group when w=1); \hat{ρ}_{W} is the estimator of correlation coefficient ρ_{W}; and

$${ρ}_{w} = \frac{Cov [ {Y}_{w} , µ ]}{\sqrt{Var [ μ ] Var [ {Y}_{w} ]}}$$

(where Y_{W} denotes potential outcomes under treatment w=1 and control w=0, σ_{W}²=Var[Y_{W}], while n₀ and n₁ are the number of enrolled control and treated subjects).

Systems configured in accordance with various embodiments of the invention may apply the above equation, relating the correlation coefficient between μᵢ and Yᵢ to the asymptotic variance of the estimate for the treatment effect. In doing so, systems may perform sample size calculations to inform RCT design. Systems may use these calculations while estimating treatment effects from randomized experiments.

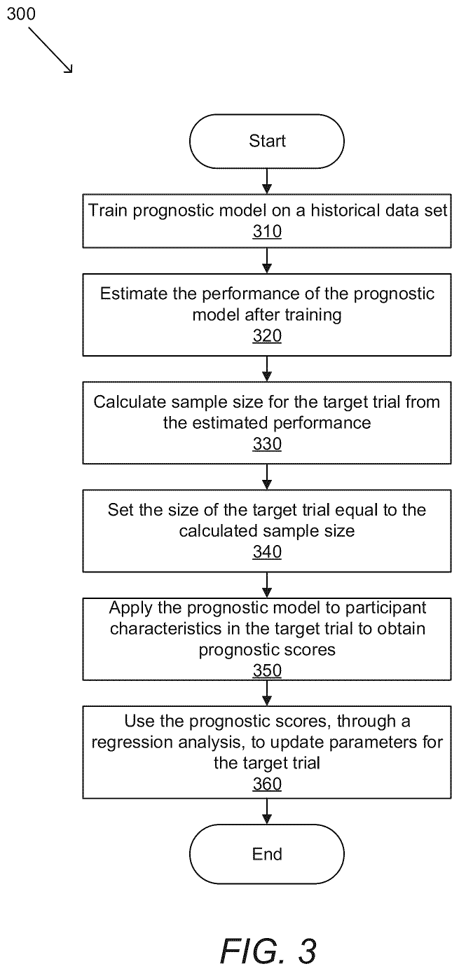

A process for accounting for historical information while estimating treatment effects from randomized experiments in accordance with certain embodiments of the invention, is illustrated in FIG. 3. In accordance with many embodiments of the invention, the process may enable systems operating in accordance with numerous embodiments to estimate treatment effects by adjusting for prognostic scores. Process 300 trains (310) a prognostic model on a historical dataset. As indicated above, historical datasets may include but are not limited to control arm data from historical control arms, patient registries, electronic health records, and real-world data.

Process 300 estimates (320) the performance of the prognostic model after training. In accordance with numerous embodiments of the invention, the performance of the invention may be represented by the squared correlation coefficient between pi and Additionally or alternatively, the subjects used in the prognostic model may utilize out-of-sample datasets that incorporate participant data similar to the makeup of the target RCT.

Process 300 calculates (330) a sample size for the target trial from the estimated performance (as disclosed above). Additional target trial parameters that may be calculated in accordance with certain embodiments include but are not limited to control arm size, and/or treatment arm size. Process 300 sets (340) the size of the target trial equal to the calculated sample size. Process 300 applies (350) the prognostic model to participant characteristics in the target trial to obtain prognostic scores. As disclosed above, in accordance with many embodiments of the invention, the prognostic model may be a digital twin used to model the expected outcome of the behavior (e.g., treatment). In such cases, the prognostic score may be represented as the mean of the expected outcome(s) determined by the digital twin (μᵢ ).

Process 300 uses (360) the prognostic scores, through a regression analysis, to update parameters for the target trial. Further, in updating the parameters while adjusting for known and unknown prognostic scores (e.g., pi, the treatment effect may thereby be estimated from the target trial using the aforementioned analysis). In accordance with numerous embodiments, regression analysis may be represented in matrix form. For example, there may be N participants in a prospective RCT such that a first vector, Y, is an N×1 vector, representing the treatment effect (i.e., outcome to be extrapolated by the regression analysis) for each participant. Additionally, Z may be an N×3 matrix with rows Zᵢ=(1, μᵢ, wᵢ ), thereby representing the vector of predictors, including but not limited to the mean of the expected outcome, and the corresponding treatment. In accordance with numerous embodiments of the invention, zᵢ∈R^{K+1}, for each participant, where K may indicate the number of predictor variables, excluding the constant predictor of “1”.

While specific processes governing the estimation of treatment effects are described above, any of a variety of processes can be utilized to account for historical information as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

In accordance with numerous embodiments of the invention, the regression formula may be represented in the following form:

Y=Zβ+∈ (D1)

This reflects why the first value of Zᵢ may be set to 1, producing the intercept term(s) α, of the regression formula (i.e., Zᵢ[1]β, also corresponding to β₀). In the above formula, ∈ may represent the random errors relative to the expected outcomes. The errors may correspond to unknown prognostic values and may take the form of, but are not limited to, vectors of random variables with currently unknown variance s². Meanwhile, β may correspond to regression coefficients used in the eventual adjustment for the unknown scores given the known (μᵢ) and unknown (∈) prognostic scores.

Using the above regression formula, an estimate for the regression coefficients may be represented as \hat{β}=(Z^{T}Z)⁻¹Z^{T}Y. Additionally or alternatively, the standard errors (i.e., precision of the sample value to the actual value) of the regression coefficients can be estimated using the sandwich estimators of the form Var[\hat{β}]=(Z^{T}Z)⁻¹Z^{T}ΩZ^{T}Z)⁻¹ in which Ω may take on different forms including but not limited to heteroskedasticity-consistent (HC) standard errors (e.g., HC0, HC1, HC2, HC3). Systems and methods in accordance with many embodiments of the invention may apply the above process to outcomes including but not limited to binary and time-to-event outcomes.

In accordance with many embodiments of the invention, in addition to refining the mean outcome μᵢ, the prognostic model disclosed in formula (A) may be used to compute the variance of the outcome:

D^{-1/2}Y=D^{-1/2}(Zβ+∈) (E)

In accordance with some embodiments, D may be a diagonal matrix with Dᵢᵢ=σ² along the diagonal. In multiplying both the left and right sides of the regression formula (Formula (D1)) by D^{-1/2}, systems and methods configured in accordance with some embodiments may reflect the following formula:

D^{-1/2}Y=D^{-1/2}(Zβ+∈) (D2)

In accordance with numerous embodiments of the invention, Formula (D2) may be modified in order to produce a function in terms of the residual (i.e., a least squares objective function). Translating this to the least squares objective function can produce

L(β)=∥D^{-1/2}(Y−Zβ)∥², (F)

which may correspond to weighting each of the squared errors by the inverse of σᵢ². As disclosed below, under methods including but not limited to Fixed and Fitted Function Weighted PROCOVA methods, the weighted least squares estimate for the regression coefficients may be,

\hat{β}_{σ}=(Z^{T}D⁻¹Z)⁻¹Z^{T}D⁻¹Y (G)

while the estimates of the covariance matrix of the weighted least squares estimator may take the form:

Vâr [\hat{β}_{σ}]=(Z^{T}D⁻¹Z)⁻¹Z^{T}D⁻¹ΩD⁻¹Z(Z^{T}D⁻¹Z)⁻¹ (H)

in which Ω depends on the choice of heteroscedasticity-consistent estimator as in the unweighted case disclosed above. As suggested above, within Z, zᵢ[1, μᵢ, Wᵢ] may represent the i^{th} row of Z, corresponding to the i^{th} participant.

In accordance with several embodiments of the invention, the expected value of the squared residuals obtained with an ordinary least squares (OLS) estimator (in the infinite sample limit) for z₁ may be represented as the skedastic function s²(zᵢ)=E[∈ᵢ²|zᵢ]. As such, in accordance with several embodiments of the invention, differing values for Ω are disclosed in Romano, J. P. and Wolf, M. (2017) “Resurrecting weighted least squares,” Journal of Econometrics, 197(1), pp. 1-19, Available at: https://doi.org/10.1016/j.jeconom.2016.10.003, incorporated by reference in its entirety. Differing values for Ω may include but are not limited to:

$${Ω}_{\frac{1}{{σ}^{2}}} : = E [ \frac{1}{{σ}^{2} ( {z}_{i} )} {z}_{i}^{T} {z}_{i} ] \qquad ( I 1 )$$

$$E [ \frac{1}{{σ}_{i}^{2}} ( \begin{matrix}1 & {μ}_{i} & {w}_{i} \\ {μ}_{i} & {μ}_{i}^{2} & {μ}_{i} {w}_{i} \\ {w}_{i} & {μ}_{i} {w}_{i} & {w}_{i}^{2}\end{matrix} ) ] \qquad ( I 2 )$$

$${Ω}_{\frac{{s}^{2}}{{σ}^{4}}} := E [ \frac{{s}^{2} ( {z}_{i} )}{{σ}^{4} ( {z}_{i} )} {z}_{i}^{T} {z}_{i} ] \qquad ( J 1 )$$

$$E [ \frac{{s}_{i}^{2}}{{σ}_{i}^{4}} ( \begin{matrix}1 & {μ}_{i} & {w}_{i} \\ {μ}_{i} & {μ}_{i}^{2} & {μ}_{i} {w}_{i} \\ {w}_{i} & {μ}_{i} {w}_{i} & {w}_{i}^{2}\end{matrix} ) ] \qquad ( J2 )$$

While, when:

$$M = E [ ( \begin{matrix}1 & {μ}_{i} & {w}_{i} \\ {μ}_{i} & {μ}_{i}^{2} & {μ}_{i} {w}_{i} \\ {w}_{i} & {μ}_{i} {w}_{i} & {w}_{i}^{2}\end{matrix} ) ] \qquad ( K )$$

the above values for Ω may thereby be estimated as:

$${Ω}_{\frac{1}{{σ}^{2}}} \approx E [ \frac{1}{{σ}^{2}} ] M and \qquad ( I 3 )$$

$${Ω}_{\frac{{s}^{2}}{{σ}^{4}}} \approx E [ \frac{{s}_{i}^{2}}{{σ}_{i}^{4}} ] M \qquad ( J 3 )$$

In accordance with many embodiments of the invention, the asymptotic distribution of the weighted least squares estimator may be:

\sqrt{N}(\hat{β}_{σ}−β)→N(0, Θ_{σ}) (L)

in which:

$${Θ}_{σ} := {Ω}_{\frac{1}{{σ}^{4}}}^{- 1} {Ω}_{\frac{{s}^{2}}{{σ}^{4}}} {Ω}_{\frac{1}{{σ}^{2}}}^{- 1} \qquad ( M )$$

Systems configured in accordance with numerous embodiments of the invention may assume zero correlation between wᵢ and μᵢ, σᵢ², and sᵢ². In particular, systems in accordance with some embodiments of the invention may assume 1) that

$$\frac{1}{{σ}^{2}}$$

is uncorrelated to μᵢ, μᵢ², wᵢ, and μᵢwᵢ; (2) that s² is uncorrelated to μᵢ, μᵢ², wᵢ, and μᵢwᵢ; and/or (3) that s²/σ⁴ is uncorrelated to μᵢ, μᵢ², wᵢ, and μᵢwᵢ. Additionally or alternatively, systems may determine that treatment is assigned at random and/or at common variance across control and treatment arms, in a manner that may be consistent with other model assumptions. Additionally or alternatively, systems may assume zero correlation between μᵢ and the two variance parameters (σᵢ², and sᵢ²). In accordance with numerous embodiments, while some association between μᵢ and σᵢ² may be expected, higher variances may be equally common at both lower and higher extremes of μᵢ such that the correlation remains zero even though some association exists.

With these approximations, the asymptotic covariance matrix of the regression coefficients may be represented as:

$${Θ}_{σ} : = \frac{E [ \frac{{s}_{i}^{2}}{{σ}_{i}^{4}} ]}{{E [ \frac{1}{{σ}_{i}^{2}} ]}^{2}} {M}^{- 1} \qquad ( N 1 )$$

Additionally or alternatively, σᵢ² may be ignored by performing an unweighted regression. In such cases, the asymptotic covariance matrix may be converted to.

Θ:=E[sᵢ²]M⁻¹ (N2)

Therefore, systems in accordance with many embodiments of the invention may follow an approximation for the asymptotic covariance matrix of the regression coefficients, wherein the matrix follows Θ_{σ}=ηΘ Tie in which:

$$η = \frac{E [ \frac{{s}_{i}^{2}}{{σ}_{i}^{4}} ]}{{E [ \frac{1}{{σ}_{i}^{2}} ]}^{2} E [ {s}_{i}^{2} ]} \qquad ( O )$$

Systems and techniques directed towards weighted regression for the assessment of uncertainty, in accordance with many embodiments of the invention, are not limited to use within Randomized Experiments. Accordingly, it should be appreciated that applications described herein can be implemented outside the context of medical trials and experiments and in contexts unrelated to prognostic models. Moreover, any of the systems and methods described herein with reference to FIG. 2 can be utilized within any of the randomized experiment arrangements described above.

### 2. Fixed Function Weighted PROCOVA

Systems and methods configured in accordance with numerous embodiments of the invention may be applied to Prognostic Covariate Adjustments (PROCOVA) processes. PROCOVA methodology is disclosed in Unlearn.AI, Inc., “PROCOVA™ Handbook for the Target Trial Statistician.” Ver. 1.0, European Medicines Agency, incorporated herein by reference. In accordance with many embodiments of the invention, Fixed Function Weighted PROCOVA methods may be configured to use historical data and prognostic modelling to decrease the uncertainty in treatment effect estimates. Systems and methods in accordance with certain embodiments of the invention, when following PROCOVA processes, may involve ordinary least squares regression analysis of RCTs. Additionally or alternatively, PROCOVA processes may use heteroskedasticity-consistent (HC) standard errors to quantify the uncertainty associated with treatment effect estimators. Heteroskedastic variables may specifically be derived in response to non-constant variance among participants to RCTs.

As indicated above, prognostic models may correspond, but are not limited, to AI-generated digital twin implementations. Examples of statistics (i.e., predictors) for digital twins may include but are not limited to means (μᵢ), standard deviations (σᵢ²), and quantiles of the distribution.

Fixed Function Weighted PROCOVA may refer to a type of weighted linear regression methodology for modelling the relationships between a response variable and a vector of predictor variables. In accordance with numerous examples, this methodology may be used when the standard assumption of constant variance in linear regression is not valid, and/or when systems intend to account for relationships between the predictor variables and the variance of the outcomes when analyzing the data.

In accordance with various embodiments of the invention, Fixed Function Weighted PROCOVA can be viewed as Zero Trust and/or Limited Trust AI solutions. Systems may thereby yield unbiased estimators for treatment effects, control Type I error rates, and/or maintain confidence interval coverage.

Fixed Function Weighted PROCOVA processes may build on PROCOVA processes through finding the coefficients β that minimize a weighted least squares loss function. As such, systems and methods configured in accordance with some embodiments may obtain samples of Monte Carlo (MC) draws from digital twins and/or utilize summaries of MC draws to implement Fixed Function Weighted PROCOVA processes. Fixed Function Weighted PROCOVA processes may utilize weighted least squares estimators obtained by finding the coefficients that minimize a weighted least squares loss function in the target RCT:

$$L ( β ) = {\sum }_{i = 1}^{N} {{C}_{i} ( {Y}_{i} - {X}_{i}^{T} β )}^{2} \qquad ( P )$$

wherein N denotes the total number of participants in the RCT, Xᵢ∈R^{K+1} denotes the vector of predictors that are used in the model for the expected outcome (making it an equivalent to vector Zᵢ in the “General Implementation” section), and β∈R^{K+1} denotes the vector of regression coefficients for the expected outcome. As such, the first entry in Xᵢ may be the number 1, corresponding to the intercept term β₀ of the regression model.

Linear regression models for outcomes produced by weighted PROCOVA processes may take the form:

Yᵢ=β₀+β₁wᵢ+β₂μᵢ+∈ᵢ (Q)

where the ∈ᵢ may distributed as independent N(0, sᵢ²) random variables. Additionally or alternatively, these error terms may not be identically distributed. The unknown variances for a specific participant i's outcome sᵢ² may thereby be modeled as a function of σᵢ² (i.e., sᵢ²=g(σᵢ²), wherein g( )represents a “skedastic function model” configured in accordance with various embodiments of the invention). For example, one skedastic function model may take the form:

g(σᵢ²)=γ₀+γ₁σᵢ² (R)

where γ₀ and γ₁ are parameters that are pre-specified (e.g., γ₀=0, γ₁=1) and/or inferred from historical data independent of the RCT data.

In accordance with various embodiments, identifying the coefficients that minimize the weighted least squares loss function may be based on fixed weights Cᵢ=1/g (σᵢ²). As such, weights as utilized by Fixed Function Weighted PROCOVA processes may be inversely proportional to the output of the skedastic function model. Additionally or alternatively, weights may be based on other predictors including but not limited to the predicted tail-area probabilities or other metrics of uncertainty in the model's predictions. Further, from these weighted values, systems may compute heteroskedasticity-consistent (HC) standard errors.

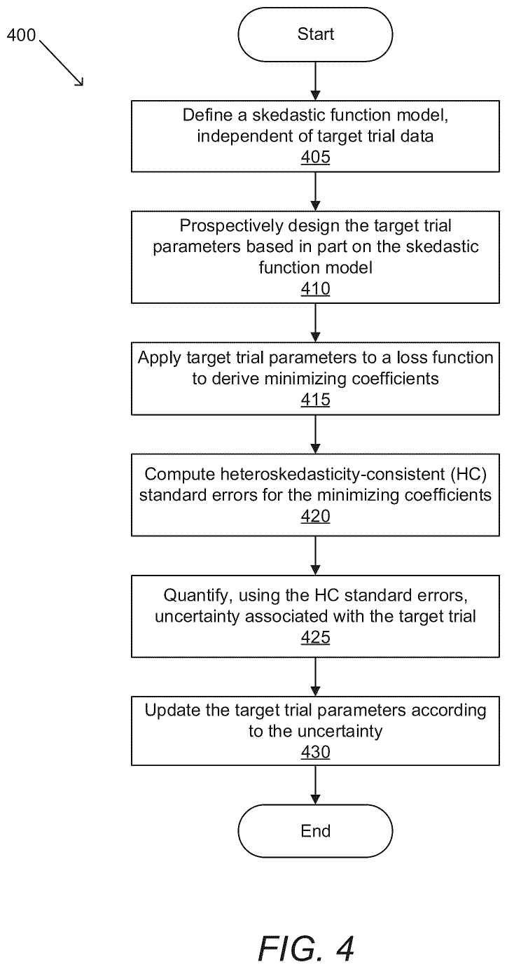

A process for Fixed Function Weighted Prognostic Covariate Adjustments, in accordance with a number of embodiments of the invention, is conceptually illustrated in FIG. 4. Process 400 defines (405) a skedastic function model, independent of the target trial data. Such target trials may include but are not limited to randomized controlled trials (RCTs). In accordance with many embodiments of the invention, skedastic function models may be defined through determining a particular mathematical form for the skedastic function model. In accordance with some embodiments, the initial form may follow standard PROCOVA models as disclosed above. In accordance with multiple embodiments, skedastic function models may or may not be defined independently of any historical data. When defined based on historical data, process 400 may define the function, at least in part, using the process disclosed in FIG. 3. Additionally or alternatively, process 400 may further define the skedastic function model using the following subprocess:

(1) fit standard PROCOVA models to the historical dataset by finding values of β (regression coefficients) that minimize the sum of squares for the standard PROCOVA models in the following formula.

$${\sum }_{i = 1}^{N} {( {Y}_{i} - {X}_{i}^{T} β )}^{2} \qquad ( S 1 )$$

As such, the estimated value for β may take the form:

$$\hat{β} = {( {X}^{T} X )}^{- 1} {X}^{T} Y where \qquad ( S 2 )$$

$$X = ( \begin{matrix}{X}_{1}^{T} \\ {X}_{2}^{T} \\ ⋮ \\ {X}_{N}^{T}\end{matrix} ) , Y = ( {Y}_{1} , … , {Y}_{N} ) .$$

(2) For each participant i, process 400 may calculate a predicted outcome in the form:

Ŷᵢ=Xᵢ^{T}\hat{β} (S3)

From the predicted outcome, process 400 may calculate residuals from the predicted and expected (Yᵢ) outcomes:

eᵢ=Yᵢ−Ŷᵢ (S4)

(3) Model the squared residuals eᵢ² in terms of a vector of predictors (Vi) including predictors of interest. In doing so, process 400 may obtain minimizing (skedastic function model parameter) values, corresponding to the coefficient minimizing vector of parameter values that minimizes the sum of squares for the modelled squared residuals. One example of modelling squared residuals may take the form:

eᵢ²=Vᵢ^{T}65 =γ₀+γ₁σᵢ² (S5)

where Vᵢ=(1, σᵢ²) and the minimizing skedastic function model parameter, γ=(γ₀, γ₁). The estimate may therefore be the vector of parameter values that minimizes the sum of squares, as in the reorganized:

$${\sum }_{i = 1}^{N} {( {e}_{i}^{2} - {γ}_{0} - {γ}_{1} {σ}_{i}^{2} )}^{2} \qquad ( T )$$

Wherein the estimate for the minimizing skedastic function model parameter vector would be) \hat{γ}=(V^{T}V)⁻¹V^{T}E, where:

$$V = ( \begin{matrix}{V}_{1}^{T} \\ {V}_{2}^{T} \\ ⋮ \\ {V}_{N}^{T}\end{matrix} ) , E = ( {e}_{1}^{2} , … , {e}_{N}^{2} ) .$$

(4) Set the skedastic function model, wherein the weights of the model are a function of \hat{γ} (e.g., g(σᵢ²)=\hat{γ}₀+\hat{γ}₁σᵢ²).

Process 400 prospectively designs (410) the target trial parameters based in part on the skedastic function model. In accordance with numerous embodiments of the invention, the design for the target trial may be specified based on mathematical formulae and/or computer code for reduction of the expected variance of the treatment effect estimator (under the Fixed Function Weighted PROCOVA method described above). The expected variance reduction may yield values for the reduction of the control arm sample size (and thereby the design) for the target trial. Additionally or alternatively, the expected variance reduction may yield a value for the power (of the test of the treatment effect). In accordance with some embodiments of the invention target trial parameters may include but are not limited to data from and/or for the target trial.

Process 400 applies (415) target trial parameters to a loss function to derive minimizing coefficients. In accordance with certain embodiments of the invention, values for the expected variance reduction in this step may be obtained in multiple ways:

(A) When the skedastic function model is pre-specified as g(σᵢ²)=σᵢ², the mathematical formula for the estimated variance reduction may be:

$$1 - \frac{E ( \frac{{s}_{i}^{2}}{{σ}_{i}^{4}} )}{{E ( \frac{1}{{σ}_{i}^{2}} )}^{2} E ( {s}_{i}^{2} )} \qquad ( U )$$

wherein the unknown values sᵢ², can be replaced by estimates obtained from the historical data above.

(B1) When the skedastic function model itself is estimated from historical control data, the mathematical formula for the estimated variance reduction may be derived from the matrices Ω_{1/g}⁻¹Ωₛ_{²}_{/g}²Ω_{1/g}⁻¹ and Ω⁻¹Ωₛ_{²}Ω⁻¹, where:

$${Ω}_{1 / g} = E ( \frac{1}{g ( {σ}_{i}^{2} )} {X}_{i} {X}_{i}^{T} ) , {Ω}_{{s}^{2} / {g}^{2}} = E ( \frac{{s}_{i}^{2}}{{g ( {σ}_{i}^{2} )}^{2}} {X}_{i} {X}_{i}^{T} ) ,$$

$$Ω = E ( {X}_{i} {X}_{i}^{T} ) , {Ω}_{{s}^{2}} = E ( {s}_{i}^{2} {X}_{i} {X}_{i}^{T} ) .$$

Specifically, when the diagonal entry in Ω_{1/g}⁻¹Ωₛ_{²}_{/g}²Ω_{1/g}⁻¹ that corresponds to the treatment indicator in Xᵢ is d1, and the diagonal entry in Ω⁻¹Ωₛ_{²}Ω⁻¹ that corresponds to the treatment indicator in Xᵢ is d2, the estimated variance reduction may take the value d1/d2. As the sᵢ² are unknown, these values can be replaced by estimates obtained from the historical data. Additionally or alternatively, the variance reduction of (Fixed Function) Weighted PROCOVA compared to (standard) PROCOVA may be represented as the following:

1−(1+CV(C)²)(1+ρₑ_{²}_{, C}²)CV(e²)CV(C²) (V)

where CV(C) denotes the coefficient of variation of the weights Cᵢ, ρₑ_{²}_{,C}² denotes the correlation between the residuals from the ordinary least squares fit and the square of the weights, and CV(e²)denotes the coefficient of variation of the residuals from the ordinary least squares fit.

(B2) Alternatively, when the skedastic function model itself is estimated from historical control data, applying (415) target trial parameters to a loss function may additionally involve the following (computational) procedure:
(1) Create a new “treatment” variable in the historical control data.
(2) Repeatedly assign the participants in the dataset to the “treated” group or the “control” group based on a pre-specified design.
(3) Specify a corresponding outcome for each participant under each assignment based on the assumed treatment effect.
(4) Implement both Fixed Function Weighted PROCOVA and PROCOVA to the data, including the treatment indicator as a predictor, under each assignment.
(5) Record the regression coefficient estimate associated with the treatment indicator under each assignment.
(6) Repeat Steps (3)-(5) many times.
(7) Calculate the variances of the regression coefficient estimates obtained from Fixed Function Weighted PROCOVA and/or PROCOVA, respectively, across the random treatment assignments.
(8) Determine the variance reduction of Fixed Function Weighted PROCOVA with respect to PROCOVA.

Once values for expected variance are obtained, they may be used to derive weights in order to perform the weighted least squares procedure as discussed above. Once the skedastic function model is defined, weights may be set to Cᵢ=1/g(σᵢ²) and used to solve for the values of β that minimize the loss function (i.e., L(β)=Σᵢ₌₁^{N}Cᵢ(Yᵢ−Xᵢ^{T}β)²). Systems and methods configured in accordance with multiple embodiments of the invention may consider additional variants of weights defined based on transformations of the squared residuals. Examples may include but are not limited to logarithmic transformations and square root transformations of the squared residuals. In such cases, models may be fit on the transformed space of the squared residuals. Additionally or alternatively, the models may be back-transformed to obtain transformed models on the original space of the squared residuals. In accordance with many embodiments of the invention, weights Cᵢ may be required to be positive, while transformations may be configured to enforce this constraint. Additionally or alternatively, the transformation that is applied to the squared residuals may also be applied to σᵢ² for model interpretability.

Process 400 computes (420) heteroskedasticity-consistent (HC) standard errors for the minimizing coefficients. Additional prospective steps for computing HC standard errors are disclosed in Romano, J. P. and Wolf, M. (2017) “Resurrecting weighted least squares,” Journal of Econometrics, 197(1), pp. 1-19, Available at: https://doi.org/10.1016/j.jeconom.2016.10.003, incorporated by reference in its entirety. Process 400 quantifies (425), using the standard errors, uncertainty associated with the target trial. Process 400 updates (430) the target trial parameters according to the uncertainty. In accordance with many embodiments of the invention, prognostic model estimates for treatment effects and estimates for their uncertainty can be used to perform hypothesis tests in order to create decision rules that guide target trials.

Skedastic function models configured in accordance with some embodiments of the invention may have no significant relationships between the predictors in the Zᵢ and the squared residual eᵢ². In such cases, all of the entries in γ that correspond to the predictors in the Zᵢ (excluding the intercept term) may effectively be estimated as zero from the historical dataset. The only non-zero coefficient may then be the intercept term γ₀, thereby making the Fixed Function Weighted PROCOVA equivalent to a PROCOVA with a constant variance.

### 3. Fitted Function Weighted PROCOVA

In accordance with numerous embodiments of the invention, Fitted Function Weighted PROCOVA methods may be configured to use RCT data and prognostic modelling to decrease the uncertainty in treatment effect estimates. In accordance with many embodiments of the invention, the RCT data used in Fitted Function Weighted PROCOVA methods (also referred to as “target trial data” in this section) may be derived directly from RCTs. Systems and methods in accordance with various embodiments of the invention, when following PROCOVA processes, may involve ordinary least squares regression analysis of the RCTs. Systems may thereby yield, asymptotically, unbiased estimators for treatment effects, control Type I error rates, and/or maintain confidence interval coverage.

In accordance with some embodiments of the invention, Fitted Function PROCOVA processes may follow many of the restrictions described under General and Fixed Function Weighted PROCOVA methods. For example, these processes may use HC standard errors to quantify the uncertainty associated with treatment effect estimators. Further, as indicated above, prognostic models may correspond, but are not limited, to AI-generated digital twin implementations. Examples of statistics (i.e., predictors) for digital twins may include but are not limited to means (μᵢ), standard deviations (σᵢ²), and quantiles of the distribution. In accordance with various embodiments of the invention, Fitted Function Weighted PROCOVA can be viewed as Zero Trust and/or Limited Trust AI solutions.

In accordance with a number of embodiments of the invention, Fitted Function Weighted PROCOVA processes may diverge from Fixed Function Weighted PROCOVA processes in relation to the use of the skedastic functions used. For example, one skedastic function model may also take the form:

g(σᵢ²)=γ₀+γ₁σᵢ². (W)

Nevertheless, in accordance with several embodiments of the invention, and in contrast to Fixed Function Weighted PROCOVA processes, parameters γ₀ and γ₁ may be inferred in manners dependent on the data for the target trial.

Systems configured in accordance with some embodiments may obtain samples of Monte Carlo (MC) draws from digital twins and/or utilize summaries of MC draws to implement Fitted Function Weighted PROCOVA processes. Additionally or alternatively, Fitted Function Weighted PROCOVA processes may utilize weighted least squares estimators obtained by finding the coefficients that minimize the weighted least squares loss function disclosed above in the target RCT:

$$L ( β ) = {\sum }_{i = 1}^{N} {{C}_{i} ( {y}_{i} - {X}_{i}^{T} β )}^{2} . \qquad ( X )$$

In accordance with various embodiments, identifying the coefficients that minimize the weighted least squares loss function may be based on fixed weights Cᵢ=1/g(σᵢ²). Additionally or alternatively, Cᵢ may be limited to positive quantities and used to derive HC standard errors. Additionally or alternatively, weight coefficients (Cᵢ) may be derived from processes including but not limited to predicted tail-area probabilities or other metrics of uncertainty in the model's predictions.

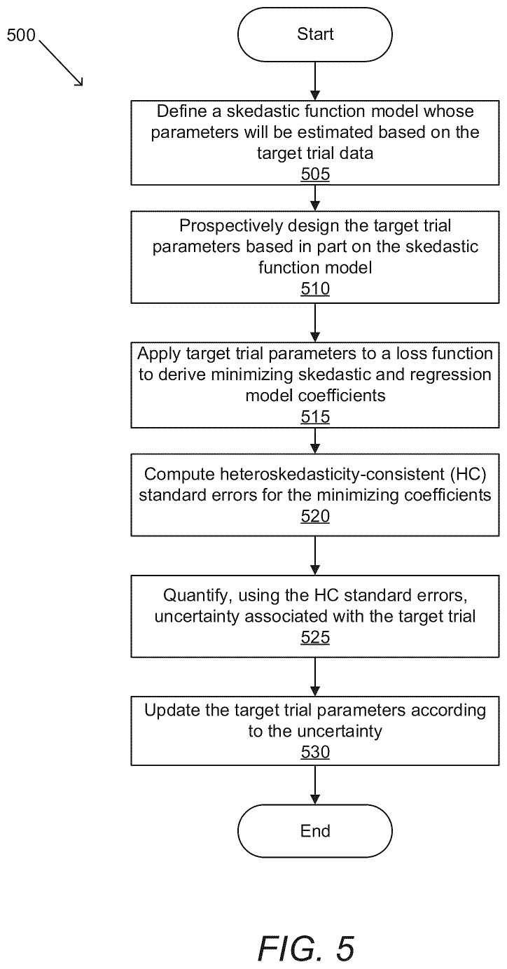

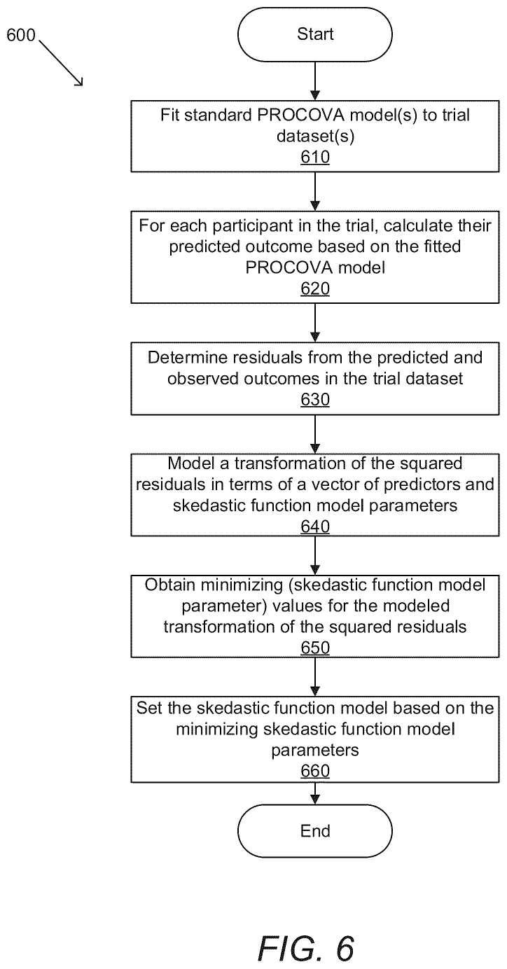

A process for Fitted Function Weighted Prognostic Covariate Adjustments, in accordance with a number of embodiments of the invention, is conceptually illustrated in FIG. 5. Process 500 defines (505) a skedastic function model, dependent on target trial data. Such target trials may include but are not limited to randomized controlled trials (RCTs). In accordance with many embodiments of the invention, skedastic function models may be defined through determining a particular mathematical form for the skedastic function model. In accordance with some embodiments, the initial form may follow standard PROCOVA models as disclosed above. Process 500 may, additionally or alternatively, define the skedastic function model using the process disclosed in FIG. 6.

Process 500 prospectively designs (510) the target trial parameters based in part on the skedastic function model. In accordance with numerous embodiments of the invention, the design for the target trial may be specified based on mathematical formulae and/or computer code for reduction of the expected variance of the treatment effect estimator. The expected variance reduction may yield values for the reduction of the control arm sample size (and thereby the design) for the target trial. Additionally or alternatively, the expected variance reduction may yield a value for the power (of the test of the treatment effect). In accordance with some embodiments of the invention target trial parameters may include but are not limited to data from and/or for the target trial.

Process 500 applies (515) target trial parameters to a loss function to derive minimizing skedastic model and minimizing regression model coefficients. In accordance with certain embodiments of the invention, values for the expected variance reduction in this step may be obtained in multiple ways:

(A) In accordance with certain embodiments, [symbol] (σᵢ²) may represent the limit of the fitted skedastic function model as N→∞. In such cases, the mathematical formula for the estimated variance reduction may be derived from the matrices [symbol] and Ω⁻¹Ωₛ_{²}Ω⁻¹, where:

$${Ω}_{1 / 𝒢 ( {σ}_{i}^{2} )} = E ( \frac{1}{𝒢 ( {σ}_{i}^{2} )} {X}_{i} {X}_{i}^{T} ) , {Ω}_{{s}^{2} / {𝒢 ( {σ}_{i}^{2} )}^{2}} = E ( \frac{{s}_{i}^{2}}{{𝒢 ( {σ}_{i}^{2} )}^{2}} {X}_{i} {X}_{i}^{T} ) ,$$

$$Ω = E ( {X}_{i} {X}_{i}^{T} ) , {Ω}_{{s}^{2}} = E ( {s}_{i}^{2} {X}_{i} {X}_{i}^{T} ) .$$

Specifically, when the diagonal entry in [symbol] that corresponds to the treatment indicator in Xᵢ is d1, and the diagonal entry in Ω⁻¹Ωₛ_{²}Ω⁻¹ that corresponds to the treatment indicator in Xᵢ is d2, the estimated variance reduction may take the value d1/d2. As the sᵢ² are unknown, these values can be replaced by estimates obtained from other, out-of-sample datasets including but not limited to datasets from other trials. These estimates may, in turn, be used to estimate what the variance reduction would be in the target trial.

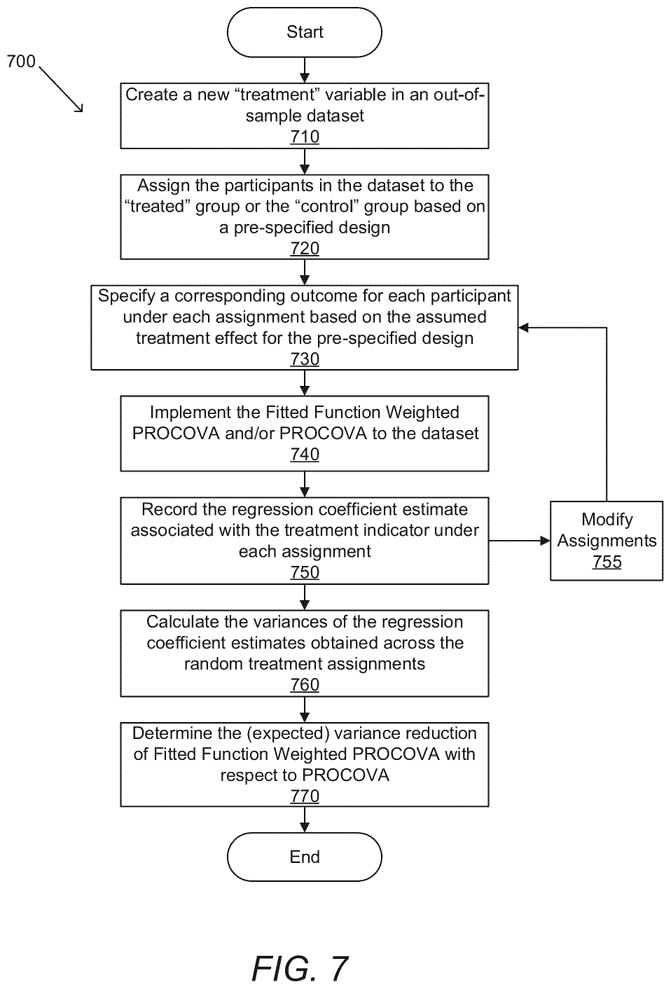

(B) Additionally or alternatively, when the skedastic function model itself is estimated from target trial data, applying (515) target trial parameters to a loss function may, additionally or alternatively, involve the (computational) procedure disclosed in FIG. 7.

Once values for expected variance are obtained, they may be used to derive weights in order to perform the weighted least squares procedure as discussed above. Once the skedastic function model is defined, weights may be set to Cᵢ=1/g(σᵢ²) and used to solve for the values of β that minimize the loss function (i.e., L(β)=Σᵢ₋₁^{N}Cᵢ(Yᵢ−Xᵢ^{T}β)²). Systems and methods configured in accordance with multiple embodiments of the invention may consider additional variants of weights defined based on transformations of the squared residuals. Examples may include but are not limited to logarithmic transformations and square root transformations of the squared residuals. In such cases, models may be fit on the transformed space of the squared residuals. Additionally or alternatively, the models may be back-transformed to obtain transformed models on the original space of the squared residuals. In accordance with many embodiments of the invention, weights Cᵢ may be required to be positive, while transformations may be configured to enforce this constraint. Additionally or alternatively, the transformation that is applied to the squared residuals may also be applied to σᵢ² for model interpretability.

Process 500 computes (520) heteroskedasticity-consistent (HC) standard errors for the minimizing coefficients. Additional prospective steps for computing HC standard errors are disclosed in Romano, J. P. and Wolf, M. (2017) “Resurrecting weighted least squares,” Journal of Econometrics, 197(1), pp. 1-19, Available at: https://doi.org/10.1016/j.jeconom.2016.10.003, incorporated by reference in its entirety.

Process 500 quantifies (525), using the standard errors, uncertainty associated with the target trial. Process 500 updates (530) the target trial parameters according to the uncertainty. In accordance with many embodiments of the invention, prognostic model estimates for treatment effects and estimates for their uncertainty can be used to perform hypothesis tests in order to create decision rules that guide target trials.

Skedastic function models configured in accordance with some embodiments of the invention may have no significant relationships between the predictors in the Zᵢ and the squared residual eᵢ². In such cases, all of the entries in γ that correspond to the predictors in the Zᵢ (excluding the intercept term) may effectively be estimated as zero from the historical dataset. The only non-zero coefficient may then be the intercept term γ₀, thereby making the Fitted Function Weighted PROCOVA equivalent to a PROCOVA with a constant variance.

A process for defining skedastic function models, in accordance with multiple embodiments of the invention, is illustrated in FIG. 6. Process 600 fits (610) standard PROCOVA model(s) to trial dataset(s). Process 600 may fit standard PROCOVA models to a trial dataset by finding values of α (regression coefficients) that minimize the sum of squares for the standard PROCOVA models in the following formula:

$${\sum }_{i = 1}^{N} {( {Y}_{i} - {X}_{i}^{T} β )}^{2} . \qquad ( Y 1 )$$

As such, the estimated value for β may take the form:

$$\hat{β} = {( {X}^{T} X )}^{- 1} {X}^{T} Y where \qquad ( Y 2 )$$

$$X = ( \begin{matrix}{X}_{1}^{T} \\ {X}_{2}^{T} \\ ⋮ \\ {X}_{N}^{T}\end{matrix} ) , Y = ( {Y}_{1} , … , {Y}_{N} ) .$$

For each participant i in the trial process 600 calculates (620) their predicted outcome based on the fitted PROCOVA model. In accordance with many embodiments, predicted outcomes may be derived from the formula:

Ŷᵢ=Xᵢ^{T}\hat{β}. (Y3)

Process 600 determines (630) residuals from the predicted and observed (Yᵢ) outcomes in the trial dataset:

eᵢ=Yᵢ−Ŷᵢ. (Y4)

Process 600 models (640) a transformation of the squared residuals eᵢ² in terms of a vector of predictors (Vᵢ), including predictors of interest, and skedastic function model parameters. In doing so, process 600 obtains (650) minimizing (skedastic function model parameter) values for the modeled transformation of the squared residuals. The minimizing (skedastic function model parameter) values may correspond to the coefficient minimizing vector of parameter values that minimizes the sum of squares for the modelled squared residuals. One example of modelling transformations of the squared residuals may take the form:

eᵢ²=Vᵢ^{T}γ=γ₀+γ₁σᵢ² (Y5)

where Vᵢ=(1, σᵢ²) and the minimizing skedastic function model parameter, γ=(γ₀, γ₁). The estimate may therefore be the vector of parameter values that minimizes the sum of squares, as in the reorganized:

$${\sum }_{i = 1}^{N} {( {e}_{i}^{2} - {γ}_{0} - {γ}_{1} {σ}_{i}^{2} )}^{2} \qquad ( Z )$$

wherein the estimate for the minimizing skedastic function model parameter vector would be) \hat{γ}=(V^{T}V)⁻¹V^{T}E, where:

$$V = ( \begin{matrix}{V}_{1}^{T} \\ {V}_{2}^{T} \\ ⋮ \\ {V}_{N}^{T}\end{matrix} ) , E = ( {e}_{1}^{2} , … , {e}_{N}^{2} ) .$$

Process 600 sets (660) the skedastic function model based on the minimizing skedastic function model parameters. In doing so, the weights of the model may be a function of \hat{γ} (e.g., g(σᵢ²)=\hat{γ}₀+\hat{γ}₁σᵢ²).

A process for obtaining expected variance reductions in accordance with certain embodiments of the invention is illustrated in FIG. 7. Process 700 creates (710) a new “treatment” variable in an out-of-sample dataset. The out-of-sample dataset may be limited to only control subjects. Process 700 repeatedly assigns (720) the participants in the dataset to the “treated” group or the “control” group based on a pre-specified design. Process 700 specifies (730) a corresponding outcome for each participant under each assignment based on the assumed treatment effect. Process 700 implements (740) the Fitted Function Weighted PROCOVA and/or PROCOVA to the dataset. In implementing (740) this, under each assignment, process may include the treatment indicator(s) as predictor(s). Process 700 records (750) the regression coefficient estimate associated with the treatment indicator under each assignment. Process 700 may repeat the implementation (740) and recordation (750) multiple times across random treatment assignments and modify (755) the assignments. Process 700 calculates (760) the variances of the regression coefficient estimates obtained from Fitted Function Weighted PROCOVA and/or PROCOVA, respectively, across the random treatment assignments. Process 700 determines (770) the (expected) variance reduction of Fitted Function Weighted PROCOVA with respect to PROCOVA.

Systems and techniques directed towards weighted regression for the assessment of uncertainty, in accordance with many embodiments of the invention, are not limited to use within PROCOVA paradigms. Accordingly, it should be appreciated that applications described herein can be implemented in contexts including but not limited to various prognostic modelling configurations and in contexts unrelated to PROCOVA paradigms and/or medical trials, such as optimization in tellurene nanomanufacturing. Moreover, any of the systems and methods described herein with reference to FIG. 5 can be utilized within any of the randomized experiment arrangements described above.

### F. Estimating the Treatment Effect Using Generative Models

In many embodiments, processes can estimate treatment effects by training two new generative models: a treatment model using the data from the treatment group, p_{θ}_{_{J}}₁(w₁), and a control model using the data from the control group, p_{θ}_{_{J}}₀(w₀). In a variety of embodiments, full panels of data from an RCT can be used to train generative models to create panels of generated data. Such processes can allow for the analysis of many outcomes (including (but not limited to) primary, secondary, and exploratory efficacy endpoints as well as safety information) by comparing the trained treatment models against trained control models. For simplicity, the notation p(y, x₀) will be used instead of p(X), with the understanding that the former can always be obtained from the latter by generating a panel of data X and then computing a specific outcome y=ƒ(X) from the panel.

In one embodiment, generative models for the control condition (e.g., a Conditional Restricted Boltzmann Machine) can be trained on historical data from previously completed clinical trials. Then, two new generative models for the control and treatment groups can be obtained by solving minimization problems:

$${min}_{{θ}_{{J}_{0}}} \{ - {\sum }_{i} ( 1 - {w}_{i} ) log {p}_{{θ}_{{J}_{0}}} ( {Y}_{i} , {x}_{0 , i} \vert {w}_{0} ) + {λ}_{0} D ( {p}_{{θ}_{{J}_{0}}} , {p}_{{θ}_{J}} ) \}$$

$${min}_{{θ}_{{J}_{1}}} \{ - {\sum }_{i} {w}_{i} log {p}_{{θ}_{{J}_{1}}} ( {Y}_{i} , {x}_{0 , i} \vert {w}_{0} ) + {λ}_{1} D ( {p}_{{θ}_{{J}_{1}}} , {p}_{{θ}_{J}} ) \}$$

in which λ₀ and λ₁ are prior parameters that describe how well pre-trained generative models describe the outcomes in the two arms of the RCT, and D(⋅,⋅) is a measure of the difference between two generative models such as (but not limited to) the Kullback-Leibler divergence. For example, the new generative models may also be Conditional Restricted Boltzmann Machines.

The estimate for the treatment effect can then be computed as

\hat{τ}=∫dy dxyp_{θ}_{_{J}}₁(y,x|w₁)−∫dy dxyp_{θ}_{_{J}}₀(y, x|w₀) (15)

In several embodiments, treatment effects can be computed by drawing samples from the control and treatment models and comparing the distributions of the samples. Processes in accordance with some embodiments of the invention can further tune the computation of treatment effects by adjusting for the uncertainty in treatment effect estimates. In several embodiments, the uncertainty in treatment effect estimates (σ_{\hat{τ}}) can be obtained using a bootstrap by repeatedly resampling the data from the RCT (with replacement), training the updated generative models, and computing an estimate for the treatment effect; the uncertainty is the standard deviation of these estimates. In a number of embodiments, point estimates for the treatment effect and the estimate for its uncertainty can be used to perform a hypothesis test in order to create a decision rule.

In numerous embodiments, processes can begin with a distribution π(θ_{J}) for the parameters of the generative model (e.g., obtained from a Bayesian analysis of historical data). Then, posterior distributions for θ_{J}_{₀} and θ_{J}_{₁} can be estimated by applying Bayes rule,

$$log π ( {θ}_{{J}_{0}} ) = constant + {\sum }_{i} ( 1 - {w}_{i} ) {p}_{{θ}_{{J}_{0}}} ( {Y}_{i} , {x}_{i} \vert {w}_{0} ) + {λ}_{0} log π ( {θ}_{J} ) \qquad ( 16 )$$

$$log π ( {θ}_{{J}_{1}} ) = constant + {\sum }_{i} {w}_{i} {p}_{{θ}_{{J}_{1}}} ( {Y}_{i} , {x}_{i} \vert {w}_{1} ) + {λ}_{1} log π ( {θ}_{J} ) .$$

In certain embodiments, point estimates for the treatment effect can be calculated as the mean of the posterior distribution

\hat{τ}=∫dy dxdθ_{J}_{₁}(y,x|w₁)π(θ_{J}_{₁})−∫dy dxdθ_{J}_{₀}p_{θ}_{_{J}}₀(y, x|w₀)π(θ_{J}_{₃}) (17)

where the uncertainty is the variance of the posterior distribution

δ²τ=∫dy dxdθ_{J}_{₁}y²p_{θ}_{_{J}}₁(y, x|w₁)π(θ_{J}_{₁})−∫dy dxdθ_{J}_{₀}y²p_{θ}_{_{J}}₀(y, x|w₀)π(θ_{J}_{₁})−\hat{τ}². (18)

As above, point estimates for the treatment effect and estimates for their uncertainty can be used to perform a hypothesis test in order to create a decision rule in accordance with certain embodiments of the invention. Processes in accordance with a variety of embodiments of the invention can train conditional generative models p_{θ}_{_{J}}₁(x₀, w₁) and p_{θ}_{_{J}}₀(x₀, w₀), as opposed to (or in conjunction with) joint generative models, in order to estimate treatment effects that are conditioned on the baseline covariates x₀.

It can be difficult to determine the operating characteristics of a decision rule based on these methods. Specifically, extensive simulations can be required in order to estimate the type-I error rate (i.e., the probability that an ineffective treatment would be declared to be effective) and the type-II error rate (i.e., the probability that an effective treatment would be declared ineffective). Well-characterized operating characteristics are required for many applications of RCTs and, as a result, this approach is often impractical. Generative models that rely on modern machine learning techniques are typically computationally expensive to train. As a result, using the bootstrap or Bayesian methods to obtain uncertainties required to formulate reasonable decision rules can be quite challenging.

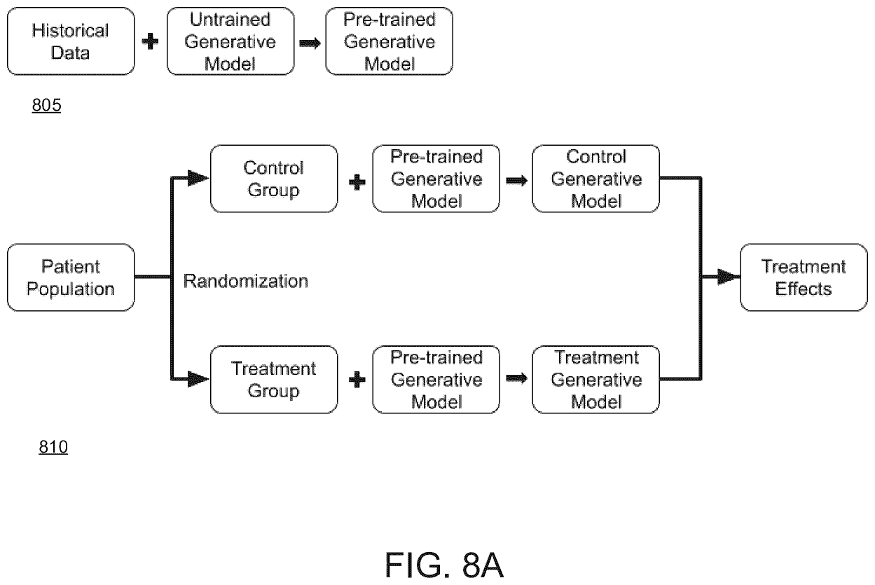

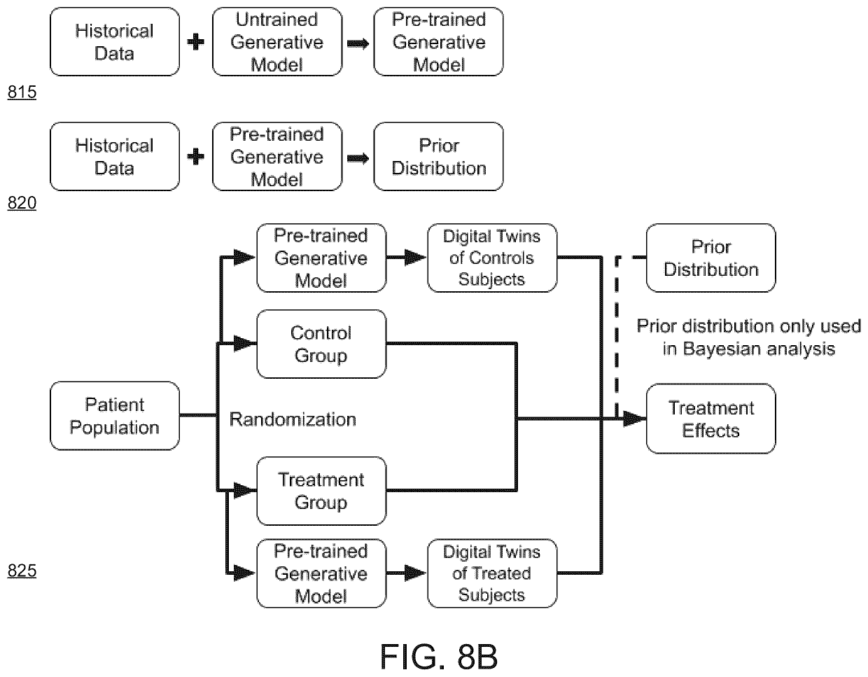

An example of using generative models to estimate treatment effects in accordance with an embodiment of the invention is illustrated in FIG. 8A. In the first stage 805, an untrained generative model of the control condition is trained using historical data, such as (but not limited to), data from previously completed clinical trials, electronic health records, and/or other studies. In the second stage 810, a patient population is randomly divided into a control group and a treatment group as part of a randomized controlled trial. Patients from the population can be randomized into the control and treatment groups with unequal randomization in accordance with a variety of embodiments of the invention. In this example, two new generative models are trained: one for the control group and one for the treatment group. In certain embodiments, control and treatment generative models can be based on a pre-trained generative model but can be additionally trained to reflect new information from the RCT. Outputs from the control and generative models can then be compared to compute the treatment effects. In several embodiments, Bayesian methods and/or the bootstrap may be used to estimate uncertainties in the treatment effects and decision rules based on p-values and/or posterior probabilities may be applied.

### G. Borrowing Information from Digital Twins

Some methods estimate treatment effects using GLMs while adjusting for covariates. For example, one may perform a regression of the final outcome in the trial against the treatment indicator and a measure of disease severity at the start of the trial. As long as the covariate was measured before the treatment was assigned in a randomized controlled trial, then adjusting for the covariate will not bias the estimate for the treatment effect in a frequentist analysis. When using covariate adjustment, the statistical power is a function of the correlation between the outcome and the covariate being adjusted for; the larger the correlation, the higher the power.

In theory, the covariate that is most correlated with the outcome that one could obtain is an accurate prediction of the outcome. Therefore, another method to incorporate generative models into RCTs in accordance with a variety of embodiments of the invention is to use generative models to predict outcomes and to adjust for the predicted outcomes in a GLM for estimating the treatment effect. Let Eₚ[yᵢ] and Varₚ[yᵢ] denote the expected value and variance of the outcome predicted for subject i by the generative model, respectively. Depending on the type of generative model, these moments may be computable analytically or, more generally, by drawing samples from the generative model p(x_{0,i}) and computing Monte Carlo estimates of the moments in accordance with a number of embodiments of the invention. The number of samples used to compute the Monte Carlo estimates can be a parameter selected by the researcher. As above, processes in accordance with several embodiments of the invention can use generative models that generate panel data so that a single generative model may be used for analyses of many outcomes in a given trial (e.g., primary, secondary, and exploratory endpoints as well as safety information). In a number of embodiments, rather than predictions for a given outcome, predictions of multiple outcomes derived from a generative model may all be included in a GLM for a particular outcome. Samples drawn from the generative models in accordance with several embodiments of the invention can be conditioned on the characteristics of a subject at the start of the trial, also referred to as digital twins of that subject.

In many embodiments, digital twins can be incorporated into an RCT in order to estimate the treatment effect by fitting a GLM of the form:

$$g ( E [ {y}_{i} ] ) = a + ( {b}_{0} + {\sum }_{j} {b}_{j} {x}_{0 , ij} ) {w}_{i} + ( {c}_{0} + {\sum }_{j} {c}_{j} {x}_{0 , ij} ) g ( {E}_{p} [ {y}_{i} ] ) + {\sum }_{j} {d}_{j} {x}_{ij} + ( {z}_{0} + {\sum }_{j} {z}_{j} {x}_{0 , ij} ) {w}_{i} g ( {E}_{p} [ {y}_{i} ] ) \qquad ( 19 )$$

in which g(⋅) is a link function. For example, g(μ)=μ corresponds to a linear regression and g(μ)=log(μ/ (1−μ)) corresponds to logistic regression. This framework in accordance with numerous embodiments of the invention can also include Cox proportional hazards models used for survival analysis as a special case. In many embodiments, some of these coefficients may be set to zero to create simpler models. One skilled in the art will recognize that it is trivial to include other predictions from the generative model as covariates if desired.

The above equation can be generalized to various applications and implementations. The terms involving the b coefficients represent the treatment effect, which may depend on the baseline covariates x₀. The terms involving the c coefficients represent potential bias in the generative model, which may depend on the baseline covariates x₀. The terms involving the d coefficients represent potential baseline differences between the treatment and control groups in the trial. The terms involving the z coefficients reflect that the relationship between the predicted and observed outcomes may be affected by the treatment. The model can be fit using any of a variety of methods for fitting GLMs. In a number of embodiments, uncertainties in the coefficients can be estimated analytically. Alternatively, or conjunctively, processes in accordance with many embodiments of the invention can estimate uncertainties using a bootstrap by repeatedly resampling the data (with replacement) and re-fitting the model; the uncertainties can be the standard deviations of the coefficients computed by this resampling procedure. In some embodiments, point estimates for the treatment effect and estimates for their uncertainty can be used to perform a hypothesis test in order to create a decision rule.

In some embodiments, variances of the outcomes can be modeled through another GLM that adjusts for the variance of the outcome that is predicted by the generative model. For example, variances in accordance with many embodiments of the invention can be modeled as follows

$$G ( Var [ {y}_{i} ] ) = α + ( {β}_{0} + {\sum }_{j} {β}_{j} {x}_{0 , ij} ) {w}_{i} + ( {γ}_{0} + {\sum }_{j} {γ}_{j} {x}_{0 , ij} ) G ( {Var}_{p} [ {y}_{i} ] ) + {\sum }_{j} {δ}_{j} {x}_{ij} + ( {ζ}_{0} + {\sum }_{j} {ζ}_{j} {x}_{0 , ij} ) {w}_{i} G ( {Var}_{p} [ {y}_{i} ] ) \qquad ( 20 )$$

in which G(⋅) is a link function that is appropriate for the variance. For example, G(σ²)=log(σ²) can be used for a continuous outcome. In many embodiments, some of these coefficients may be set to zero to create simpler models. One skilled in the art will recognize that other predictions from the generative model can be included as covariates if desired.

Well-trained generative models in accordance with certain embodiments of the invention will have g(E[yᵢ])≈(Eₚ[yᵢ]) and G(Var[yᵢ])≈G(Varₚ[yᵢ]) by construction. Therefore, prior knowledge about the coefficients in the GLMs can be used to improve the estimation of the treatment effect. However, machine learning models may not generalize perfectly to data outside of the training set. Typically, the generalization performance of a model is measured by holding out some data from the model training phase so that the held-out data can be used to test the performance of the model. For example, suppose that there are one or more control arms from historical clinical trials in addition to the generative model. Then, the c coefficients in accordance with various embodiments of the invention can be estimated by fitting a reduced GLM on the historical control arm data,

$$g ( E [ {y}_{i} ] ) = a + ( {c}_{0} + {\sum }_{j} {c}_{j} {x}_{0 , i j} ) g ( {E}_{p} [ {y}_{i} ] ) , \qquad ( 21 )$$

for the mean or

$$G ( Var [ {y}_{i} ] ) = α + ( {γ}_{0} + {\sum }_{j} {γ}_{j} {x}_{0 , i j} ) G ( {Var}_{p} [ {y}_{i} ] ) , \qquad ( 22 )$$

for the variance. This is particularly useful in a Bayesian framework, in which a distribution π(a, c) or π(α, γ) can be estimated for these coefficients using the historical data, where the data-driven prior distribution can be used in a Bayesian analysis of the RCT. Essentially, this uses the historical data to determine how well the generative model is likely to generalize to new populations, and then applies this information to the analysis of the RCT. In the limit that π(a, c)→δ(a−0)δ(c−1), then digital twins in accordance with a variety of embodiments of the invention can become substitutable for actual control subjects in the RCT. As a result, the better the generative model, the fewer control subjects required in the RCT. In some embodiments, similar approaches could be used to include prior information on any coefficients that are active when wᵢ=0, including the d coefficients.

Examples of workflows for frequentist and Bayesian analyses of clinical trials that incorporate digital twins to estimate treatment effects in accordance with various embodiments of the invention are described below. For a frequentist case for a continuous endpoint, consider a simple example

E[yᵢ]=a+b₀wᵢ+c₀Eₚ[yᵢ] (23)

Var[yᵢ]=σ² (24)

assuming no interactions and homoscedastic errors. One skilled in the art will recognize how this can be applied to the more general case captured by Equation 19 and Equation 20. In numerous embodiments, simple analyses can lead to results that are more easily interpreted. This model implies a normal likelihood,

yᵢ˜N(a+b₀wᵢ+c₀Eₚ[yᵢ], σ²) (25)

such that the model can be fit (e.g., by maximum likelihood). There are two situations to consider: (1) the design of the trial has already been determined by some method prior to incorporating the digital twins such that the digital twins can be used to increase the statistical power of the trial, or (2) the trial needs to be designed so that it incorporates digital twins to achieve an efficient design with sufficient power. In the case of a continuous endpoint, the statistical power of the trial will depend on the correlation between yᵢ and Eₚ[yᵢ], which can be estimated from historical data, and is a function of the magnitude of the treatment effect. In a variety of embodiments, analytical formulas can be derived in this special case. Alternatively, or conjunctively, computer simulations can be utilized in the general case.

Once the trial is designed, patients are enrolled and followed until their outcome is measured. In some cases, patients may not be able to finish the trial and various methods (such as Last Observation Carried Forward) need to be applied in order to impute outcomes for the patients who have not finished the trial, as in most clinical trials. In a number of embodiments, GLMs can be fit to the data from the trial to obtain point estimates \hat{b}₀ and uncertainties \hat{σ}_{b}_{₀} for the treatment effect. The ratio \hat{b}₀/\hat{σ}_{b}_{₀} follows a Student's t-distribution which can be used to compute a p-value p_{b}_{₀} and the null-hypothesis that there is no treatment effect can be rejected if p_{b}_{₀}≤ [symbol] in which [symbol] is the desired control of the type-I error rate. This approach is guaranteed to control the type-I error rate, whereas the realized power will be related to the out-of-sample correlation of yᵢ and Eₚ[yᵢ] and the true effect size.

In the Bayesian case for a continuous endpoint with homoscedastic errors, assume a simple analysis,

E[yᵢ]=a+b₀wᵢ+c₀Eₚ[yᵢ] (26)

Var[yᵢ]=σ² (27)

In certain embodiments, the simple analysis can lead to results that are more easily interpreted. This model implies a normal likelihood,

yᵢ˜N(a+b₀wᵢ+c₀Eₚ[yᵢ], σ²) (28)

but processes in accordance with various embodiments of the invention can use a Bayesian approach to fit it instead of the method of maximum likelihood. In particular, with historical data representing the condition wᵢ=0 that was not used to train the generative model, processes in accordance with many embodiments of the invention can fit the model,

E[yᵢ]=a+c₀Eₚ[yᵢ] (29)

to the historical data in order to derive prior distributions for the analysis of the RCT. To do so, pick a prior distribution π(a, c₀, σ²) such as (but not limited to) a Normal-Inverse-Gamma prior or another appropriate prior distribution. As there are no data to inform the parameters of the prior before analyzing the historical data, processes in accordance with several embodiments of the invention can use a diffuse or default prior. In numerous embodiments, Bayesian updates to the prior distribution can be computed from the historical data to derive a new distribution π_{H}(a, c₀, σ²), in which the subscript H can be used to denote that this distribution was obtained from historical data. Processes in accordance with numerous embodiments of the invention can then specify a prior distribution π₀(b₀) for the treatment effect. This could also be derived from data in accordance with many embodiments of the invention if it's available, or a diffuse or default prior could be used. The full prior distribution is now π_{H}(a, c₀, σ²)π₀(b₀). In various embodiments, such distributions can be used to compute the expected sample size in order to design the trial, as in a typical Bayesian trial design. Once the trial is designed, patients can be enrolled and followed until their outcome is measured. In some cases, patients may not be able to finish the trial and various methods (such as Last Observation Carried Forward) can be applied in order to impute outcomes for the patients who have not finished the trial, as in most clinical trials.

In numerous embodiments, GLMs can be fit to obtain a posterior distribution π_{RCT}(a, b₀, c₀, σ²) for the parameters. A point estimate for the treatment effect can be computed by, for example, \hat{b}₀=∫da db₀ dc₀ dσ²b₀π_{RCT}(a, b₀, c₀, σ²); though, other Bayesian point estimates could be computed as well. In several embodiments, the posterior probability that the treatment effect is greater than zero can also be computed as Prob(b₀≥0)=∫da db₀ dc₀ dσ²θ(b₀≥0)π_{RCT}(a, b₀, c₀, σ²), in which θ(·) is a logic function that returns one if the argument is true and zero otherwise. As in a typical Bayesian analysis, the treatment can be declared effective if Prob(b₀≥0) exceeds a pre-specified threshold in accordance with a number of embodiments of the invention.

There are two limits to the Bayesian analysis that can be informative. First, in the limit of a flat prior distribution π_{H}(a, c₀, σ²)π₀(b₀)∝1, then the point estimate and uncertainty for the treatment effect will converge to give the same results as the maximum likelihood method described previously. Thus, if the generalizability of the digital twin model to the population in the RCT is questionable then the Bayesian analysis will end up being very similar to the frequentist analysis. In contrast to the method used to estimate a treatment effect in a trial including digital subjects, including digital twins in the analysis still leads to a gain in power as long as yᵢ is correlated with Eₚ[yᵢ]. The other instructive limit is π_{H}(a, c₀, σ²)π₀(b₀)∝δ(a−0)δ(c₀−1). In this limit, the point estimate for the treatment effect converges to \hat{b}₀=N_{T}⁻¹ Σᵢ (yᵢ−Eₚ[yᵢ])wᵢ. That is, in some embodiments, the estimate for the treatment effect can be obtained by taking the average of the difference between observed and predicted outcomes for the patients who received the treatment wᵢ=1. Notice that this latter prior distribution can lead to a situation in which the data from the patients who received the control treatment wᵢ=0 can be ignored. Processes in accordance with various embodiments of the invention can run trials without a concurrent control arm.

There are advantages and disadvantages to the frequentist and Bayesian methods that are captured through these simple examples. The frequentist approach to including digital twins in the analysis of an RCT leads to an increase in statistical power while controlling the type-I error rate. If desired, it's also possible to use the theoretical increase in statistical power to decrease the number of subjects required for the concurrent control arm, although this cannot be reduced to zero concurrent control subjects. The Bayesian approach borrows more information about the generalizability of the model used to create the digital twins (e.g., from an analysis of historical data) and, as a result, can increase the power much more than the frequentist approach. In addition, the use of Bayesian methods in accordance with numerous embodiments of the invention can enable one to decrease the size of the concurrent control arm even further. However, the increase in power/decrease in required sample size can come at the cost of an uncontrolled type-I error rate. Therefore, processes in accordance with many embodiments of the invention can perform computer simulations of the Bayesian analysis to estimate the type-I error rate so that the operating characteristics of the trial can be described.

As a final example, it is helpful to consider a simple case in which a GLM is also used for the variance. For example, consider the model

E[yᵢ]=a+b₀wᵢ+c₀Eₚ[yᵢ] (30)

logVar[yᵢ]=α+β₀wᵢ+γ₀logₚVar[yᵢ], (31)

which reflect the likelihood:

yᵢ˜N(a+b₀wᵢ+c₀Eₚ[yᵢ], e^{α+β}^{₀}^{w}^{ᵢ}^{+γ}^{₀}^{logVar}^{ₚ}^{[y}^{ᵢ}]). (32)

Models in accordance with a number of embodiments of the invention can allow for heteroscedasticity in which the variance of the outcome is correlated with the variance predicted by the digital twin model, and in which the variance may be affected by the treatment. In several embodiments, a system of GLMs can be fit (e.g., using maximum likelihood, Bayesian approaches, etc.), as was the case for the simpler model. One skilled in the art will clearly recognize that one could also include the interaction or other terms in order to model more complex relationships if necessary. In addition, one skilled in the art will also recognize that including interactions can lead to estimates of conditional average treatment effects in addition to average treatment effects.

An example of borrowing information from digital twins to estimate treatment effects in accordance with an embodiment of the invention is illustrated in FIG. 8B. In the first part 815, a generative model of the control condition is trained using historical data from previously completed clinical trials, electronic health records, or other studies. In the second part 820, if the analysis to be performed is Bayesian, predictions from the generative model are compared to historical data that were not used to train the model in order to obtain a prior distribution capturing how well the predictions generalize to new populations. A frequentist analysis does not need to obtain a prior distribution. In the third part 825, a randomized controlled trial is conducted (potentially with unequal randomization), digital twins are generated for each subject in the trial, and all of the data are incorporated into a statistical analysis (including the prior from step 820 if the analysis is Bayesian) to estimate the treatment effects. Bayesian methods, analytical calculations, or the bootstrap may be used to estimate uncertainties in the treatment effects, and decision rules based on p-values or posterior probabilities may be applied.

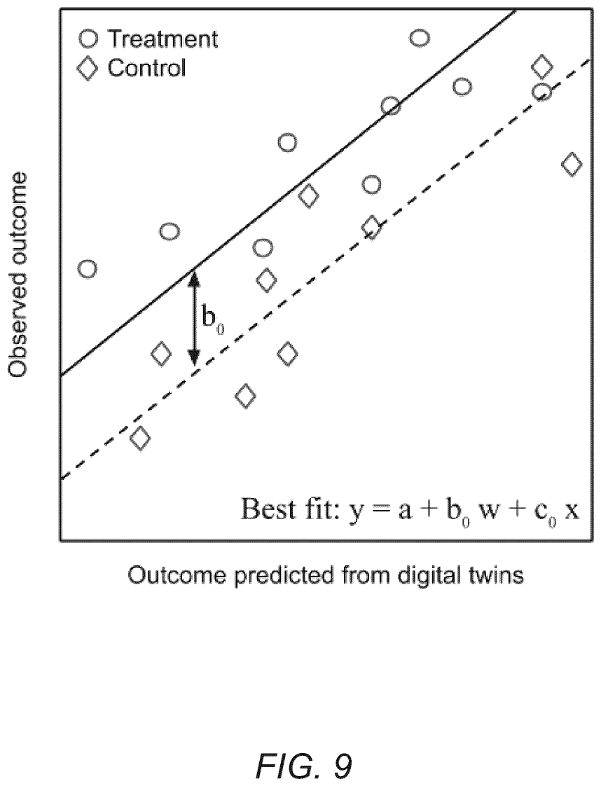

An example of using linear models and digital twins to estimate treatment effects in accordance with an embodiment of the invention is illustrated in FIG. 9. This drawing illustrates the concept using a simple analysis of a continuous outcome. The x-axis represents the prediction for the outcome from the digital twins, and the y-axis represents the observed outcome of the subjects in the RCT. A linear model is fit to the data from the RCT, adjusting for the outcome predicted from the digital twins. If no interactions are included, then two parallel lines are fit to the data: one to the control group and one to the treatment group. The distance between these lines is an estimate for the treatment effect. Both frequentist and Bayesian methods may be used to analyze the generalized linear model.

### H. Systems for Determining Treatment Effects

### 1. Treatment Analysis System

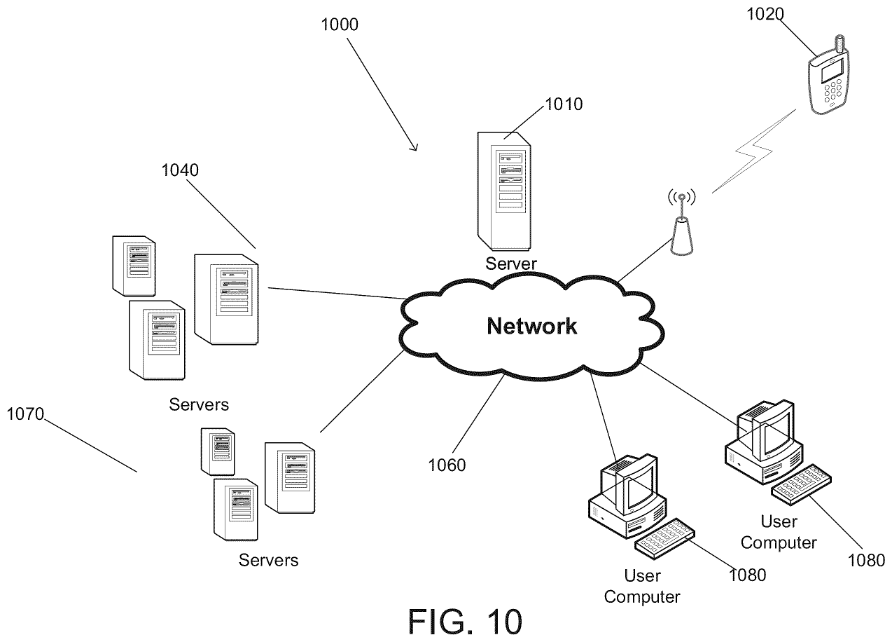

An example of a treatment analysis system that determines treatment effects in accordance with some embodiments of the invention is illustrated in FIG. 10. Network 1000 includes a communications network 1060. The communications network 1060 is a network such as the Internet that allows devices connected to the network 1060 to communicate with other connected devices. Server systems 1010, 1040, and 1070 are connected to the network 860. Each of the server systems 1010, 1040, and 1070 is a group of one or more servers communicatively connected to one another via internal networks that execute processes that provide cloud services to users over the network 1060. One skilled in the art will recognize that a treatment analysis system may exclude certain components and/or include other components that are omitted for brevity without departing from this invention.

For purposes of this discussion, cloud services are one or more applications that are executed by one or more server systems to provide data and/or executable applications to devices over a network. The server systems 1010, 1040, and 1070 are shown each having three servers in the internal network. However, the server systems 1010, 1040 and 1070 may include any number of servers and any additional number of server systems may be connected to the network 1060 to provide cloud services. In accordance with various embodiments of this invention, treatment analysis systems in accordance with various embodiments of the invention may be provided by a process being executed on a single server system and/or a group of server systems communicating over network 1060.

Users may use personal devices 1080 and 1020 that connect to the network 1060 to perform processes that determine treatment effects in accordance with various embodiments of the invention. In the shown embodiment, the personal devices 1080 are shown as desktop computers that are connected via a conventional “wired” connection to the network 1060. However, the personal device 1080 may be a desktop computer, a laptop computer, a smart television, an entertainment gaming console, and/or any other device that connects to the network 1060 via a “wired” connection. The mobile device 1020 connects to network 1060 using a wireless connection. A wireless connection is a connection that uses Radio Frequency (RF) signals, Infrared signals, and/or any other form of wireless signaling to connect to the network 1060. In FIG. 10, the mobile device 1020 is a mobile telephone. However, mobile devices 1020 may be mobile phones, Personal Digital Assistants (PDAs), tablets, smartphones, and/or any other type of device that connects to network 1060 via wireless connection without departing from this invention.

As can readily be appreciated the specific computing system used to determine treatment effects is largely dependent upon the requirements of a given application and should not be considered as limited to any specific computing system(s) implementation.

### 2. Treatment Analysis Element

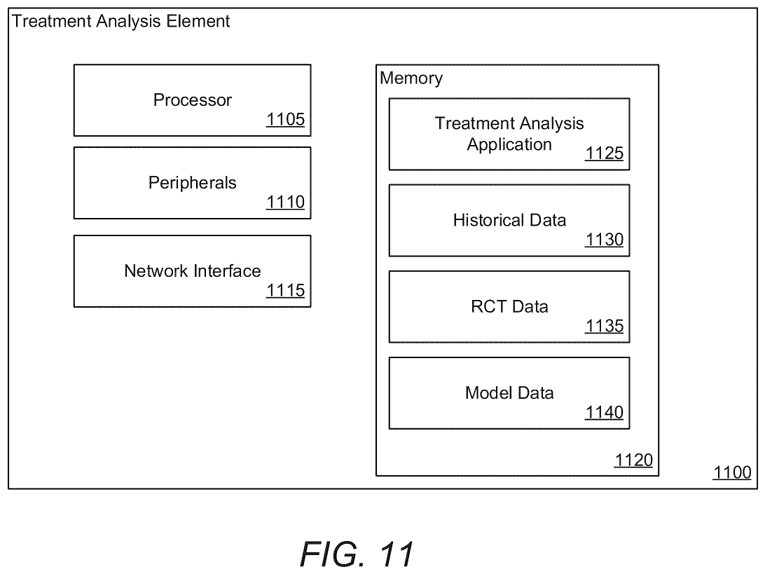

An example of a treatment analysis element that executes instructions to perform processes that determine treatment effects in accordance with various embodiments of the invention is illustrated in FIG. 11. Treatment analysis elements in accordance with many embodiments of the invention can include (but are not limited to) one or more of mobile devices, cloud services, and/or computers. Treatment analysis element 1100 includes processor 1105, peripherals 1110, network interface 1115, and memory 1120. One skilled in the art will recognize that a treatment analysis element may exclude certain components and/or include other components that are omitted for brevity without departing from this invention.

The processor 1105 can include (but is not limited to) a processor, microprocessor, controller, and/or a combination of processors, microprocessor, and/or controllers that performs instructions stored in the memory 1120 to manipulate data stored in the memory. Processor instructions can configure the processor 1105 to perform processes in accordance with certain embodiments of the invention.

Peripherals 1110 can include any of a variety of components for capturing data, such as (but not limited to) cameras, displays, and/or sensors. In a variety of embodiments, peripherals can be used to gather inputs and/or provide outputs. Treatment analysis element 1100 can utilize network interface 1115 to transmit and receive data over a network based upon the instructions performed by processor 1105. Peripherals and/or network interfaces in accordance with many embodiments of the invention can be used to gather data that can be used to determine treatment effects.

Memory 1120 includes a treatment analysis application 1125, historical data 1130, RCT data 1135, and model data 1140. Treatment analysis applications in accordance with several embodiments of the invention can be used to determine treatment effects of an RCT, to design an RCT, and/or determine decision rules for treatments.

Historical data in accordance with many embodiments of the invention can be used to pre-train generative models to generate potential outcomes for digital subjects and/or digital twins. In numerous embodiments, historical data can include (but is not limited to) control arms from historical control arms, patient registries, electronic health records, and/or real-world data. In many embodiments, predictions from the generative model can be compared to historical data that were not used to train the model in order to obtain a prior distribution capturing how well the predictions generalize to new populations.

In some embodiments, RCT data can include panel data collected from subjects of a RCT. RCT data in accordance with a variety of embodiments of the invention can be divided into control and treatment arms based on whether subjects received a treatment. In many embodiments, RCT data can be supplemented with generated subject data. Generated subject data in accordance with a number of embodiments of the invention can include (but is not limited to) digital subject data and/or digital twin data.

In several embodiments, model data can store various parameters and/or weights for generative models. Model data in accordance with many embodiments of the invention can include data for models trained on historical data and/or trained on RCT data. In several embodiments, pre-trained models can be updated based on RCT data to generate digital subjects.

Although a specific example of a treatment analysis element 1100 is illustrated in this figure, any of a variety of treatment analysis elements can be utilized to perform processes for determining treatment effects similar to those described herein as appropriate to the requirements of specific applications in accordance with embodiments of the invention.

### 3. Treatment Analysis Application

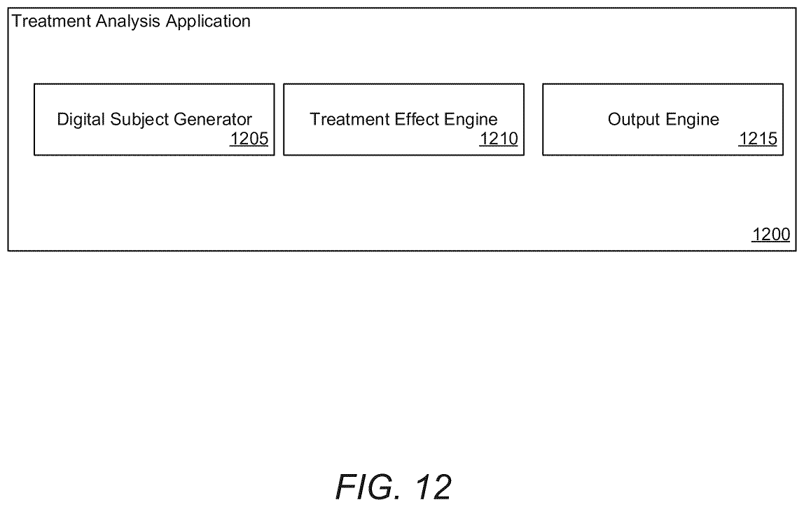

An example of a treatment analysis application for determining treatment effects in accordance with an embodiment of the invention is illustrated in FIG. 12. Treatment analysis application 1200 includes digital subject generator 1205, treatment effect engine 1210, and output engine 1215. One skilled in the art will recognize that a treatment analysis application may exclude certain components and/or include other components that are omitted for brevity without departing from this invention.

Digital subject generators in accordance with various embodiments of the invention can include generative models that can generate digital subject and/or digital twin data. Generative models in accordance with certain embodiments of the invention can be trained to generate potential outcome data based on characteristics of an individual and/or a population. Digital subject data in accordance with several embodiments of the invention can include (but is not limited to) panel data, outcome data, etc. In several embodiments, generative models can include (but are not limited to) traditional statistical models, generative adversarial networks, recurrent neural networks, Gaussian processes, autoencoders, autoregressive models, variational autoencoders, and/or other types of probabilistic generative models.

In various embodiments, treatment effect engines can be used to determine treatment effects based on generated digital subject data and/or data from a RCT. In some embodiments, treatment effect engines can use digital subject data from digital subject generators to determine a treatment effect in a variety of different applications, such as, but not limited to, comparing separate generative models based on data from the control and treatment arms of a RCT, supplementing a control arm in an RCT, comparing predicted potential control outcomes with actual treatment outcomes, etc. Treatment effects engines in accordance with some embodiments of the invention can be used to determine individualized responses to treatment. In certain embodiments, treatment effect engines can determine biases of generative models of the digital subject generator and incorporate the biases (or corrections for the biases) in the treatment effect analyses.

Output engines in accordance with several embodiments of the invention can provide a variety of outputs to a user, including (but not limited to) decision rules, treatment effects, generative model biases, recommended RCT designs, etc. In numerous embodiments, output engines can provide feedback when the results of generative models of a digital subject generator diverge from the RCT population. For example, output engines in accordance with certain embodiments of the invention can provide a notification when a difference between generated control outcomes for digital twins of subjects from a control arm and their actual control outcomes exceeds a threshold.

Although a specific example of a treatment analysis application is illustrated in this figure, any of a variety of Treatment analysis applications can be utilized to perform processes for determining treatment effects similar to those described herein as appropriate to the requirements of specific applications in accordance with embodiments of the invention.

Systems and techniques for applying prognostic models to assessment of experiment uncertainty, are not limited to use for randomized controlled trials. Accordingly, it should be appreciated that applications described herein can be implemented outside the context of generative model architecture and in contexts unrelated to RCTs. Moreover, any of the systems and methods described herein with reference to FIGS. 1-12 can be utilized within any of the generative models described above.

Although specific methods of determining treatment effects are discussed above, many different methods of treatment analysis can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for estimating treatment effects for a target trial, the method comprising:
defining a skedastic function model, wherein defining the skedastic function model depends, at least in part, on trial data that was applied in a trial;
designing trial parameters for a target trial based in part on the skedastic function model;
applying the trial parameters to a loss function to derive at least one minimizing outcome coefficient, wherein the at least one minimizing outcome coefficient corresponds to a regression coefficient for an expected outcome to the target trial based on the trial parameters;
computing standard errors for the at least one minimizing outcome coefficient;
quantifying, using the standard errors, values for uncertainty associated with the target trial; and
updating the trial parameters according to the uncertainty.

2. The method of claim 1, wherein the standard errors are heteroskedasticity-consistent standard errors.

3. The method of claim 1, wherein the expected outcome is obtained through at least one of the group consisting of a digital twin and a prognostic model.

4. The method of claim 1, wherein defining the skedastic function model comprises:
calculating one or more predicted outcomes for the trial data;
obtaining residuals corresponding to the one or more predicted outcomes for the trial data; and
using the residuals to define the skedastic function model.

5. The method of claim 4, wherein predicted outcomes for the trial data are based on digital twin outputs.

6. The method of claim 5, wherein:
the predicted outcomes are predictions from a regression model fitted on the trial data; and
predictors of the regression model are means of the digital twin outputs.

7. The method of claim 4, wherein the trial data comprises participant data for an RCT.

8. The method of claim 4, wherein defining the skedastic function model further comprises:
applying parameters of the skedastic function model to a loss function for data from the target trial, to derive at least one minimizing model coefficient, wherein the at least one minimizing model coefficient includes a treatment effect coefficient;
computing standard errors for the at least one minimizing model coefficient;
calculating one or more predicted outcomes for the target trial; and
defining the skedastic function model further based on variances corresponding to the one or more predicted outcomes for the target trial.

9. The method of claim 8, wherein:
predicted outcomes for the target trial are based on digital twin outputs; and
minimizing model coefficients are treatment effect coefficients.

10. The method of claim 1, wherein the loss function is a weighted least squares loss function.

11. The method of claim 10, wherein at least one weight quantity of the weighted least squares loss function is inversely proportional to a predicted variance of outcomes of a participant in the target trial.

12. The method of claim 10, wherein each weight quantity of the weighted least squares loss function has a positive value.

13. The method of claim 10, wherein at least one weight quantity of the weighted least squares loss function is defined by:
implementing, using trial data, an ordinary least squares fit;
obtaining least squares coefficients from the ordinary least squares fit; and
deriving, from the least squares coefficients and the trial parameters, the at least one weight quantity.

14. The method of claim 1, wherein:
updating the trial parameters according to the uncertainty comprises determining a set of characteristics for the target trial, wherein the set of characteristics comprises a number of subjects to be enrolled in each of a control arm and a treatment arm; and
the uncertainty is based on at least one of a desired type-I error rate and a desired type-II error rate.

15. The method of claim 1, wherein updating the trial parameters comprises at least one of:
minimizing a total number of samples for at least one selected from the group consisting of a treatment arm of the target trial, a control arm of the target trial, and the target trial in totality; and
performing a regression analysis based on the expected outcome.

16. The method of claim 15, wherein:
an estimate for coefficients of the regression analysis is represented as:
\hat{β}=(Z^{T}Z)⁻¹Z^{T}Y
Y is a vector corresponding to treatment outputs for each participant; and
Z is a matrix for which each row (zᵢ) corresponds to a set of predictor variables for a participant (i).

17. The method of claim 16, wherein the set of predictor variables for each participant comprise the expected outcome and a corresponding treatment for the participant.

18. The method of claim 15, wherein minimizing a total number of samples is performed by deriving an expected variance reduction.

19. The method of claim 18, wherein deriving the expected variance reduction comprises:
obtaining a limit for the skedastic function model;
deriving a set of estimated variance reductions for the previous trial, wherein the estimated variance reduction for each participant of the previous trial is derived from a ratio between a diagonal entry of a first matrix and a diagonal entry of a second matrix; and
determining the expected variance reduction from the set of estimated variance reductions.

20. The method of claim 19, wherein:
Xᵢ is a vector of predictor variables for a participant i;
sᵢ² is a representation of the unknown outcome variance for the participant i;
the first matrix is represented as: [symbol] , where:
$${Ω}_{1 / 𝒢 ( {σ}_{i}^{2} )}^{- 1} E ( \frac{1}{𝒢 ( {σ}_{i}^{2} )} {X}_{i} {X}_{i}^{T} ) ,$$
$${Ω}_{{s}^{2} / {𝒢 ( {σ}_{i}^{2} )}^{2}} = E ( \frac{{s}_{i}^{2}}{{𝒢 ( {σ}_{i}^{2} )}^{2}} {X}_{i} {X}_{i}^{T} ) ,$$
and [symbol]
(σᵢ²) is the limit of the skedastic function model for the participant i; and
the second matrix is represented as: Ω⁻¹Ω_{S}_{²}Ω⁻¹, where:
Ω=E(XᵢXᵢ^{T}),
and
Ω_{S}_{²}=E(sᵢ²XᵢXᵢ^{T}).
